(19) United States
(12) Patent Application Publication
(10) Pub. No.: US 2023/0293578 A1
(43) Pub. Date: Sep. 21, 2023

(54) TREATMENT OF BACTERIAL VAGINOSIS

(71) Applicant: Gary A. Gelbfish, Brooklyn, NY (US)

(72) Inventor: Gary A. Gelbfish, Brooklyn, NY (US)

(21) Appl. No.: 18/017,239

(22) PCT Filed: Jul. 30, 2021
(86) PCT No.: PCT/US21/44076

§ 371 Date: Jan. 20, 2023

Related U.S. Application Data
(60) Provisional application No. 63/059,817, filed on Jul. 31, 2020.

(51) Int. Cl. A61K 33/32; A61K 38/40; A61K 33/30; A61K 38/19; A61K 31/7016; A61K 45/06; A61K 9/00; A61P 31/04

(52) U.S. Cl. CPC A61K 33/32; A61K 38/40; A61K 33/30; A61K 38/19; A61K 31/7016; A61K 45/06; A61K 9/0034; A61P 31/04

(57) ABSTRACT

The present invention provides therapeutic compositions and methods for treatment of bacterial vaginosis and restoration of the vaginal microbiome. The compositions include an active pharmaceutical ingredient a filler a binder and a gelling agent.

### REFERENCE TO RELATED APPLICATIONS

This application claims benefit of U.S. provisional patent application No. 63/059,817 entitled “Slow Release Formulations for Vaginal Medications including Treatment of Bacterial Vaginosis” filed Jul. 31, 2020, the entire contents of which are incorporated by reference herein for all purposes,

### FIELD OF THE INVENTION

This invention relates to compositions for treatment of bacterial vaginosis infection and restoration of vaginal microbiome.

### BACKGROUND OF THE INVENTION

Bacterial vaginosis (BV) is the leading vaginal disorder in women of childbearing age. Women with BV are at increased risk for acquiring of several sexually transmitted infections (STIs) including HIV, Neisseria gonorrhoeae, Chlamydia trachomatis, and herpes simplex virus 2, as well as complications after gynecologic surgery, and recurrence of BV itself. BV also increases the risk of HIV transmission to male sex partners. BV during pregnancy is an established risk factor for spontaneous abortion, preterm delivery and associated neonatal complications. Even for infants born at term, an increased risk of neonatal assisted ventilation/respiratory distress, admission to neonatal intensive care units, and neonatal sepsis was found in BV-exposed infants. Not all patients with BV are symptomatic. However, the Centers for Disease Control recommends that all symptomatic women should be treated for BV to relieve symptoms and signs of infection and to reduce the risk of acquiring and spreading HIV and other STIs.

BV is characterized by a profound shift in the normal vaginal microbiota, from lactobacilli in a low-pH environment that are replaced with multiple pathogenic species (e.g. Gardnerella vaginalis, Atopobium vaginae and Prevotella species) in an elevated pH environment. The lining of the human female vagina is normally inhabited by several Lactobacilli species that produce lactate and maintain a low pH environment. Normally, these bacteria provide a protective barrier against infection by undesirable bacteria which cannot live in the relatively acidic environment. These beneficial lactobacilli are described scientifically as the “normal vaginal microbiome.” A disturbance of this normal microbiome is the hallmark of BV. Symptomatic and asymptomatic BV are estimated to be highly prevalent in various populations with some studies showing one or more episodes in 29% of all women aged 14 to 49 years and in 50% of African American women.

Currently approved, standard-of-care treatments for BV all rely on antibiotics targeting pathogenic microbes, and all show unacceptably low cure rates and high relapse rates. While antibiotics are typically successful to provides relief of symptoms, the relief is most often temporary, and recurrence is common, with an approximately 50% recurrence within 6 months. There have been many efforts to impact this vicious cycle of treatment and recurrence, but without significant success to date. The limitations of this unidimensional antibiotic-based approach to such a complex condition are well known.

Other approaches to BV treatment include vaginal probiotics, vaginal pH manipulation, metal chelation compounds, DNases for biofilm disruption, simultaneous treatment of sexual partners with antibiotics or condom use to avoid reinfection, and contraceptive hormonal intervention. None of these interventions has proved a reliable, sustained treatment for BV.

Vaginal drug delivery has been traditionally used to deliver contraceptives and drugs to treat vaginal infections. A variety of formulations such as creams, gels, bioadhesive polymers, thermo-gelling agents, rings, suppositories, pessaries have been developed for vaginal drug delivery of contraceptives and therapeutic compositions. Bioadhesive formulations have been developed as new types of controlled-release delivery systems however, there are challenges to these delivery systems such as leakage, messiness and low vaginal residence time contributing to poor patient compliance. Current development continues to focus on formulation improvement leading to better compliance. Bacterial vaginosis (BV) is an example of a type of infection in which effective vaginal treatment formulations warrant improvement.

All of the above illustrate the need for innovative approaches for drug delivery and effective treatment for BV ideally to selectively restore and maintain the vaginal microbiome to its pre-BV healthy state.

### SUMMARY OF INVENTION

The invention is drawn to prevention and treatment of bacterial vaginosis. In an aspect the invention treats BV with selective nutritional deprivation of vaginal nutrients that are essential only to bacteria associated with BV condition, thus depressing their growth while selectively enhancing the vaginal environment with nutrients that specifically promote the growth of protective lactobacilli restoring the vaginal environment to a normal microbiome.

In another embodiment a treatment algorithm has been developed by combining our collected data with genetic, metabolic, nutritional and evolutionary aspects of the critical bacterial species in both the normal and BV microbiota.

In an embodiment a new product therapy may accomplish both eradication of pathological BV bacteria such as Gardnerella vaginalis and suppression of transitional bacteria such as Lactobacillus iners. In an embodiment the therapy helps to restore a normal microbiome. In another embodiment the therapy is a follow up therapy, following a course of antibiotics which target pathological BV bacteria but do not change L. iners, a transitional species.

The invention is drawn to treatment of BV both by iron sequestration and by vaginal supplementation of essential minerals and carbohydrates to encourage the growth of beneficial Lactobacilli and to the detriment of BV bacteria and Lactobacillus iners. It is well established that animal and human cells and bacteria need iron to live. Iron is used for critical metabolic reduction/oxidation (redox) reactions where iron is used for electron transfer. This occurs when iron transitions between its Fe2+ (ferrous) and Fe3+ (ferric) states.

In an embodiment a method of treating and preventing occurrence/recurrence of bacterial vaginosis in women comprising vaginally administering a therapeutic composition capable of sequestering iron and selectively reducing growth of bacteria associated with bacterial vaginosis.

In an embodiment the therapeutic composition selectively deprives bacteria associated with bacterial vaginosis of essential nutrients.

In an embodiment the bacteria deprived of nutrients is selected from the group consisting of L. iners, G. vaginalis, A. vaginae, M. curtisii, M. mulieris, Megaspheraera spp. and P. bivia.

In an embodiment the bacteria selected for nutrient deprivation is L. iners; a lactobacillus that is considered transitional and enhances the probability of progressing to BV.

In an embodiment, the therapeutic composition enhances growth of beneficial bacteria, restoring the normal vaginal microbiome. The beneficial bacteria is lactobacillus selected from the group consisting of L. crispatus, L. jensenii, L. gasseri, L. helveticus, L. rhamnosus, L. reuteri, L. casei and other less common but beneficial Lactobacillus subspecies that produce lactic acid in sufficient quantities to lower the vaginal pH and prevent BV.

In an embodiment the treatment decreases recurrence of bacterial vaginosis.

In an embodiment, the therapeutic composition is anti-inflammatory.

In an embodiment the therapeutic composition comprises at least one of or any combinations thereof selected from the group consisting of lactoferrin, bovine lactoferrin, apolactoferrin, manganese, zinc, copper, simple sugars (e.g. lactose), lipocalin-2 (LCN2) and probiotic.

In an embodiment the therapeutic composition is formulated as a vaginal suppository.

In an embodiment further comprising administering the therapeutic composition before, during or following a course of antibiotics.

In an embodiment a method of seeding the vaginal microbiome comprising administering a probiotic having selective compounds to enhance growth of beneficial lactobacillus species.

In an embodiment a method of seeding the vaginal microbiome comprising administering a therapeutic composition and a probiotic having selective compounds to enhance growth of beneficial lactobacillus species.

In an embodiment a composition for the treatment of bacterial vaginosis comprising at least one of or any combinations thereof selected from the group consisting of bovine lactoferrin, apolactoferrin, manganese, zinc, copper, lipocalin-2, sugars and probiotics.

In an embodiment a composition may also comprise a second active pharmaceutical ingredient selected from the group consisting of proteins, peptides, minerals, trace elements, antifungals, antibiotics, anti-inflammatories, cytokines, hormones, anesthetics, analgesics, probiotics and fragments thereof in addition to the vaginosis agent(s).

The therapeutic composition of the present invention may further comprise one or more excipients, including gelling agents, lubricants, glidants and combinations thereof. These excipients can serve one or more function for example a excipient can be both a bioadhesive gelling agent and a binder. The therapeutic composition of the present invention may include a lubricant, examples include magnesium stearate, stearic acid, or glyceryl behenate which aid in tablet manufacturing.

In a further embodiment, the therapeutic composition of the present invention can be administered before, concomitantly or following administration of an antibiotic. In the case of bacterial vaginosis, standard of care treatment is a course of antibiotics. Thus, it is envisioned that the therapeutic composition could be administered in support of this therapy, being additive or synergetic, restoring the vaginal microbiome and could possibly improve efficacy of the antibiotic treatment.

The therapeutic composition can be in any convenient physical dosage form but is preferably in the form of a pessary. For example, when treating bacterial vaginosis, the therapeutic composition is preferably in the form of a tablet which can be introduced intravaginally.

In another embodiment, a tablet may consist of two or more distinct layers, with each layer having its own blend of excipients and therapeutics and each having its own phamaco-kinetic dissolution profile depending on the dissolution needs of the particular therapeutic.

In another embodiment, a tablet may consist of two or more distinct sides, with each side having its own blend of excipients and therapeutics and each having its own phamaco-kinetic dissolution profile depending on the dissolution needs of the particular therapeutic.

In another embodiment, a kit may comprise the therapeutic compositions described above. In a specific embodiment, the kit may comprise daily active dosage units comprising a therapeutic composition, herein the therapeutic composition allows for daily dosing regimen as prescribed, and wherein the initial administration of the therapeutic composition establishes its effect on bacterial vaginosis.

In another embodiment a kit may comprise the therapeutic composition of the present invention as well as an antibiotic formulation that is standard of care, thus having the ability to provide both antibiotic and therapeutic composition for daily dosage regimen.

In an embodiment methods are provided for the use of the therapeutic compositions for the treatment of bacterial vaginosis.

In an embodiment the invention treats BV with selective nutritional deprivation of vaginal nutrients that are essential only to bacteria associated with the BV condition, thus depressing their growth. Simultaneously, it selectively enhances the vaginal environment with nutrients that specifically promote the growth of protective lactobacilli modifying the vaginal environment to only support the protective bacterial growth and help restore the normal vaginal microbiome.

In an embodiment the invention is drawn to improved slow release compositions and methods thereof for vaginal drug delivery. The present invention provides a slow release therapeutic composition comprising at least one active pharmaceutical ingredient, a binder, a filler and a gelling agent, as formulated in the therapeutic composition, presents a therapeutic effect in human hosts, either alone or in combination with other materials. Examples of pharmaceutical ingredients useful in the present invention include proteins, peptides, minerals, trace elements, antibiotics, antifungals, anti-inflammatories, cytokines, hormones, anesthetics, analgesics, probiotics and fragments thereof.

In an embodiment the slow release therapeutic composition comprises at least one active pharmaceutical ingredient, binder, filler, and a gelling agent for the treatment of bacterial vaginosis either alone or in combination with other materials. The therapeutic indication of bacterial vaginosis provides proof of concept for the slow release compositions of the invention for vaginal delivery Preferred active pharmaceutical ingredients of the present invention for the treatment of bacterial vaginosis are selected from the group consisting of lactoferrin, bovine lactoferrin, manganese, zinc, lipocalin-2, apolactoferrin, sugars or combinations thereof. The therapeutic composition of the present invention can be administered before, concomitantly or following administration of an antibiotic, probiotic or other established BV treatment method that would be enhanced by synergistic modes of action.

In an embodiment the composition is a slow release composition for delivery of the therapeutic composition. In that regard, the therapeutic effect may be due to the direct action of the pharmaceutical ingredient of the composition or may be due to one or more other materials additively or synergistically interacting with the pharmaceutical ingredient. The present invention provides a therapeutic composition delivered intravaginally for vaginal drug delivery. An example of such a therapeutic is a therapeutic composition delivered intravaginally for the treatment of bacterial vaginosis. By treatment of or treatment for bacterial vaginosis, it is meant that the therapeutic composition is effective to prevent or reduce the incidence, severity and/or duration of the infection. The therapeutic composition may also prevent reoccurrence of the infection. The treatment may be of a person not exhibiting any, exhibiting only some or exhibiting all symptoms of the condition, The treatment may be a prophylactic treatment.

A therapeutically effective amount or therapeutically effective dose of an active pharmaceutical ingredient refers to an amount of the active pharmaceutical ingredient administered as part of a treatment regimen to effect such a treatment, alone or in combination with other active pharmaceutical ingredients.

The composition may also comprise a second active pharmaceutical ingredient selected from the group consisting of proteins, peptides, minerals, trace elements, antifungals, antibiotics, anti-inflammatories, cytokines, hormones, anesthetics, analgesics, probiotics and fragments thereof in addition to the vaginosis agent(s).

The therapeutic composition of the present invention may further comprise one or more excipients, including lubricants, glidants and combinations thereof. The therapeutic composition of the present invention may include a lubricant, examples include magnesium stearate, stearic acid, or glyceryl behenate which aid in tablet manufacturing.

In an embodiment the slow release therapeutic composition comprising at least one active pharmaceutical ingredient, binder, lubricant, filler, glidant and a bioadhesive gelling agent for the treatment of bacterial vaginosis either alone or in combination with other materials.

In a further embodiment, the therapeutic composition of the present invention can be administered before, concomitantly or following administration of an antibiotic, probiotics or other established or emerging methods of treatment for BV whose success rate for treatment and recurrence can be improved by the synergy of additional mechanisms of action that our novel approach provides and that will assist to restore a normal microbiome. In the case of bacterial vaginosis, the current standard of care treatment is a course of antibiotics. Thus, it is envisioned that the therapeutic composition could be administered in support of this therapy, being additive or synergetic and could possibly improve efficacy of the antibiotic treatment. In a further embodiment, the therapeutic composition of the present invention can be used with any treatment modality wherein synergy with the treatment composition would be expected to aid in the treatment of BV and restoration of the biome.

The therapeutic composition can be in any convenient physical dosage form but is preferably in the form of a tablet of pessary. For example, when treating bacterial vaginosis, the therapeutic composition is preferably in the form of a tablet which can be introduced intravaginally. Upon contact with the low moisture environment of the vagina the outer layer of the tablet is observed to transition to a gel containing the therapeutic agents, due to the hygroscopic and gelling nature of its chosen excipients as it contacts moisture in the vagina. This gel forms a coating that surrounds the tablet and acts to prevent further fluid contact and tablet dissolution. The inner portion of the tablet remains dry and the tablet stays intact as the surface slowly erodes. Further dissolution will only occur when, over time, the gel is naturally dispersed onto the vaginal mucosa away from the tablet. Once this occurs, the therapeutic composition is slowly released and further prolongs the contact time of the tablet to the vaginal mucosa. The therapeutic composition can provide from about 12 hours to about 36 hours of dissolution in vivo. This approach contrasts with compositions that include binders that rapidly wet or disintegrants that are designed to rupture the tablet with wetting and disperse the therapeutic composition more rapidly.

In an embodiment the active pharmaceutical ingredient of the therapeutic composition is lactoferrin, and more preferably bovine lactoferrin. In one embodiment the slow release therapeutic composition comprises lactoferrin, a binder, a filler and a gelling agent that, as formulated in the therapeutic composition, presents a therapeutic effect in human hosts, for the treatment of bacterial vaginosis either alone or in combination with other materials. In that regard, the therapeutic effect may be due to the direct action of the pharmaceutical ingredient of the composition or may be due to one or more other materials synergistically interacting with the pharmaceutical ingredient. The present invention provides a therapeutic composition delivered intravaginally for the treatment of bacterial vaginosis. In a preferred embodiment the therapeutic composition comprises bovine lactoferrin, a bioadhesive polymer, a binder, lubricant, and filler.

In another embodiment the therapeutic composition containing lactoferrin contains a second active pharmaceutical ingredient for example proteins, peptides, minerals, antibiotics, anti-inflammatories, cytokines, hormones, anesthetics, analgesics, probiotics and fragments thereof.

In another embodiment the therapeutic composition containing lactoferrin can be administered concomitantly with a second active pharmaceutical ingredient for example proteins, peptides, minerals, antibiotics, anti-inflammatories, cytokines, hormones, anesthetics, analgesics, probiotics and fragments thereof.

In another embodiment the therapeutic composition containing lactoferrin can be administered before or following administration of another active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing lactoferrin can be administered before, concomitantly or following administration of an antibiotic. In an embodiment the therapeutic composition containing lactoferrin is administered following antibiotic treatment for bacterial vaginosis.

In another embodiment the therapeutic composition comprises manganese as an active pharmaceutical ingredient, a binder and a filler that, as formulated in the therapeutic composition, for the treatment of bacterial vaginosis either alone or in combination with other materials. The therapeutic composition delivered intravaginally, containing manganese in a therapeutic composition can be formulated as immediate release or a slow release composition.

Manganese can be administered concomitantly with a therapeutic composition of the present invention in which lactoferrin is the active pharmaceutical ingredient. In an embodiment manganese is added as a second active pharmaceutical ingredient to a slow release therapeutic composition containing lactoferrin, a binder, a filler and a gelling agent to additionally enhance the growth of a lactobacillus dominated vaginal microbiome.

In another embodiment the therapeutic composition containing manganese contains a second active pharmaceutical ingredient for example proteins, peptides, minerals, antibiotics, anti-inflammatories, cytokines, hormones, anesthetics, analgesics, probiotics and fragments thereof. In a preferred embodiment apolactoferrin is added as a second active pharmaceutical ingredient to a therapeutic composition containing manganese.

In another embodiment the therapeutic composition containing manganese can be administered concomitantly with a second active pharmaceutical ingredient for example proteins, peptides, minerals, antibiotics, anti-inflammatories, cytokines, hormones, anesthetics, analgesics, probiotics and fragments thereof.

In another embodiment the therapeutic composition containing manganese can be administered before or following administration of another active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing manganese can be administered before, concomitantly or following administration of an antibiotic. In an embodiment the therapeutic composition containing lactoferrin is administered following antibiotic treatment for bacterial vaginosis.

In another embodiment the active pharmaceutical ingredient is apolactoferrin, a form of lactoferrin that contains little iron (only approximately <4% of iron binding sites occupied). In another embodiment the therapeutic composition comprises apolactoferrin as an active pharmaceutical ingredient, a binder and a filler that, as formulated in the therapeutic composition, for the treatment of bacterial vaginosis either alone or in combination with other materials. The therapeutic composition delivered intravaginally, containing apolactoferrin in a therapeutic composition can be formulated as immediate release or a slow release composition. The present invention provides a slow release therapeutic composition comprising apolactoferrin as an active pharmaceutical ingredient, a binder, a filler and a gelling agent that, as formulated in the therapeutic composition delivered intravaginally for the treatment of bacterial vaginosis.

In another embodiment the therapeutic composition containing apolactoferrin contains a second active pharmaceutical ingredient for example proteins, peptides, minerals, antibiotics, anti-inflammatories, cytokines, hormones, anesthetics, analgesics, probiotics and fragments thereof.

In another embodiment the therapeutic composition containing apolactoferrin can be administered concomitantly with a second active pharmaceutical ingredient for example proteins, peptides, minerals, antibiotics, anti-inflammatories, cytokines, hormones, anesthetics, analgesics, probiotics and fragments thereof.

In another embodiment the therapeutic composition containing apolactoferrin can be administered before or following administration of another active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing apolactoferrin can be administered before, concomitantly or following administration of an antibiotic. In an embodiment the therapeutic composition containing lactoferrin is administered following antibiotic treatment for bacterial vaginosis.

In an embodiment the pharmaceutical ingredient is lipocalin-2. In an embodiment the therapeutic composition comprises lipocalin-2, as the active pharmaceutical ingredient, a binder and a filler that, as formulated in the therapeutic composition, presents a therapeutic effect in human hosts. The therapeutic composition delivered intravaginally, containing lipocalin-2 in a therapeutic composition can be formulated as immediate release or a slow release composition. The present invention provides a therapeutic composition comprising lipocalin-2, as the active pharmaceutical ingredient, a binder, a filler and a gelling agent delivered intravaginally for the treatment of bacterial vaginosis.

In another embodiment the therapeutic composition containing lipocalin-2 can be administered with a second active pharmaceutical ingredient for example proteins, peptides, minerals, antibiotics, anti-inflammatories, cytokines hormones, anesthetics, analgesics, probiotics and fragments thereof. In an embodiment apolactoferrin is the second active ingredient in the therapeutic composition.

In a further embodiment, the therapeutic composition containing lipocalin-2 can be administered concomitantly with another pharmaceutical ingredient for example proteins, peptides, minerals, antibiotics, anti-inflammatories, cytokines, hormones, anesthetics, analgesics, probiotics and fragments thereof.

In another embodiment the therapeutic composition containing lipocalin-2 can be administered before or following administration of another active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing lipocalin-2 can be administered before, concomitantly or following administration of an antibiotic. In an embodiment the therapeutic composition containing lipocalin-2 is administered following antibiotic treatment for bacterial vaginosis.

In an embodiment the therapeutic composition comprises lactoferrin, lipocalin-2, a bioadhesive gelling agent, binder, lubricant, glidant and filler that, as formulated in the therapeutic composition, presents a therapeutic effect in human hosts, for the treatment of bacterial vaginosis either alone or in combination with other materials. In that regard, the therapeutic effect may be due to the direct action of the pharmaceutical ingredient of the composition or may be due to one or more other materials synergistically interacting with the pharmaceutical ingredient. The present invention provides a therapeutic composition delivered intravaginally for the treatment of bacterial vaginosis.

In another embodiment the therapeutic compositions of the present invention may be used for treatment of preterm birth. Vaginal dysbiosis caused by BV is associated with a risk of late miscarriage or preterm birth. Inflammation is another risk factor for preterm birth, and an increased level of several inflammatory markers is typically seen in BV. Therapeutic compositions that deliver slow release levels of lactoferrin, a protein that has both iron-binding and anti-inflammatory activities, may prevent preterm birth. The therapeutic composition comprises at least one active pharmaceutical ingredient, and at least one excipient that, as formulated in the therapeutic composition, presents a therapeutic effect in human hosts, for the treatment of vaginal dysbiosis either alone or in combination with other materials. The therapeutic composition of the present invention comprises lactoferrin, a bio-adhesive polymer, and at least one excipient that, as formulated in the therapeutic composition, presents a therapeutic effect in human hosts, for the treatment of vaginal dysbiosis either alone or in combination with other materials resulting in the prevention or reduction in occurrence of pre term birth.

In another embodiment, a kit may comprise the therapeutic compositions described above. In a specific embodiment, the kit may comprise daily active dosage units comprising a pharmaceutical ingredient in a therapeutic composition, herein the therapeutic composition allows for daily dosing regimen as prescribed, and wherein the initial administration of the therapeutic composition establishes its effect on bacterial vaginosis.

In another embodiment a kit may comprise the therapeutic composition of the present invention as well as an antibiotic formulation that is standard of care, thus having the ability to provide both antibiotic and therapeutic composition for daily dosage regimen.

In another embodiment, the present disclosure provides a method for making the slow release composition comprising: combining one or more active pharmaceutical ingredients, a binder and a filler, mixing the ingredients to combine, and pressing the ingredients into a tablet. Excipients can be added to improve the desired characteristics of the tablet.

In an embodiment methods are provided for the use of the therapeutic compositions for the treatment of bacterial vaginosis.

### BRIEF DESCRIPTION OF THE DRAWINGS

FIG. 1 Panel A depicts a SDS-PAGE analysis showing levels of Difesan lactoferrin formulation and Metrodora Therapeutics bovine Lactoferrin (bLF) Z4 formulation in vaginal fluid. Panel B. shows a concentration-time profile of Difesan lactoferrin formulation in vaginal fluid. Panel C. shows a concentration-time profile of bLF Z2 slow release lactoferrin formulation in vaginal fluid.

FIG. 2 depicts a growth curve of Gardnerella vaginalis in the presence of lactoferrin.

FIG. 3 shows a bar graph of Gardnerella vaginalis growth in the presences of lactoferrin, antibiotic and the combination of lactoferrin and antibiotic.

FIG. 4 shows levels of calprotectin in healthy women and women with bacterial vaginosis, n=16, line indicates median value (22 μg/mL vs 223 μg/mL for healthy vs BV).

FIG. 5 depicts concentrations of manganese and zinc in vaginal fluid from women with unknown BV status and concentrations of calprotectin in vaginal fluid from women with healthy (L. crispatus dominated), L. iners-dominated or BV vaginal microbiomes.

FIG. 6 Panel A depicts predicted concentrations of manganese in vaginal fluid after administration of a 50 μg dose of Mn in slow release formulation. Dotted lines show min and max concentrations of endogenous Mn from n=20 women. Dashed lines show amount of Mn bound by calprotectin in BV (n=24).

FIG. 6 Panel B depicts predicted concentrations of manganese vaginal fluid after administration of a 250 μg dose of Zn in a slow release formulation. Dotted lines show min and max concentrations of endogenous Zn from n=20 women. Dashed lines show amount of Mn bound by calprotectin in BV (n=24).

### DETAILED DESCRIPTION

### Definitions

Unless defined otherwise, all technical and scientific terms used herein shall have the same meaning as commonly understood by one of ordinary skill in the art.

As used herein, “By” means bacterial vaginosis.

As used herein, “LF” means lactoferrin.

As used herein “bLF” means bovine lactoferrin.

As used herein “hLF” means human lactoferrin.

As used herein “apo” means apolactoferrin. Apolactoferrin contains little (1-2%) to no iron in contrast with native lactoferrin which contains at least approximately 15% iron. As used herein, “siderophores” refer to small, high-affinity iron-chelating compounds that are secreted by microorganisms such as bacteria and fungi and serve primarily to scavenge and transport iron across cell membranes.

As used herein “bioadhesive” means having the ability to adhere to a biological surface such as mucous membranes or other tissues for an extended period of time.

As used herein “LCN2” means lipocalin-2 a protein involved in innate immunity by sequestrating iron containing siderophores, thus limiting bacterial growth.

As used herein “slow release” refers to the release of an active pharmaceutical ingredient that occurs more slowly relative to an immediate release dosage form. The term may be used interchangeably with “sustained release”, “controlled release”, “modified release” or “extended release”.

The invention provides treatment for bacterial vaginosis and restoration of the microbiome utilizing selective nutritional deprivation of vaginal nutrients that are essential only to bacteria associated with BV condition, thus depressing their growth. Simultaneously, it selectively enhances the vaginal environment with nutrients that specifically promote the growth of protective lactobacilli thus modifying the vaginal environment to support protective bacterial growth.

The most common vaginal Lactobacillus species are L. crispatus, L. Jensenii, L. Gasseri, and L. iners. The first three species produce sufficient lactate to create an unusually low pH vaginal environment that lactobacilli thrive in and are relatively protective against BV bacterial invasion, hence will be referred to as “L. crispatus-type” bacteria. The fourth species, L. iners, produces less lactate, creates a higher pH vaginal environment and is less effective at preventing BV infection. An L. iners-based microbiome, is permissive to and can coexist with the many BV bacteria. As such, L. iners is recognized as a transitional species, that when present in significant quantities facilitates the development and recurrence of BV.

L. crispatus-type bacteria are acknowledged as the most beneficial bacteria in the vaginal microbiome, however, no one has proposed a treatment regimen that directly targets L. iners which would result in a competitive advantage and favor the growth of L. crispatus-type bacteria. Current standard of care for BV antibiotics (e.g. metronidazole, secnidazole, and clindamycin) are of no use, since both beneficial lactobacilli species and L. iners have similar sensitivities and are spared by these BV antibiotics. Indeed, following standard of care BV treatment, in which the main BV bacteria are successfully suppressed, L. iners most often outcompetes L. crispatus and returns to be the dominant species of the vaginal microbiota.

Besides therapy for the acute episode of BV infection, the ultimate long-term solution of treating BV is to develop a therapy that will encourage Lactobacillus crispatus-like growth and discourage L. iners growth, thereby restoring a protective microbiome against BV. As such, in an embodiment a therapy is provided that may accomplish suppression of both pathological BV bacteria and L. iners. In an embodiment the therapy restores a partially or fully normal microbiome. To accomplish this, we studied the nuanced differences between L. crispatus and L. iners that relate to phenotypical characteristics, metabolic pathways and nutritional requirements. It should be noted that complete restoration of the vaginal biome to pre-infection levels may not be attainable due to severity, duration and frequency of infections, the goal is to restore the microbiome wherein L. crispatus type bacteria are predominant or at least sufficiently functional to materially impact the occurrence of BV.

As initially described in 1975, nutritional immunity is a potent and ubiquitous biological mechanism whereby the host sequesters iron and deprives bacteria of their constant iron needs, in order to limit their growth. In a disease state, “iron withholding” describes the additional efforts implemented to further limit iron availability to bacteria. By limiting access to iron, the body aims to limit an overgrowth of bacteria on mucosal surfaces or invasion into the body tissues and bloodstream (sepsis.) Complex mechanisms accomplish the task of sequestering iron, including high affinity binding to complex proteins such as transferrin or lactoferrin, incorporating iron into complex structure such as ferritin and sequestering iron intracellularly via hepcidin/ferroportin signaling.

The concept of nutritional immunity was recently further expanded, when it was discovered that manganese and zinc are part of this system, as they are sequestered by the protein calprotectin. Sequestration of these metals is accomplished by these protein binding sites that are highly specific for certain metals and ionic states. This is unlike other chelators, such as EDTA that non-selectively binds many multivalent metals. It must be emphasized that while nutritional immunity does not target a specific species, it inevitably affects some bacterial species more than others, since comparatively, bacterial baseline requirements and internal bacterial reserve capacity for these metals vary. Furthermore, some bacteria have developed alternative methods which they up-regulate when iron deprived, in order to source these critical minerals despite the body's attempts at sequestration. Several known mechanisms include 1) specialized bacterial transport mechanisms to source mineral needs from low concentration environments, 2) specialized bacterial binding proteins that directly extract iron from chelation proteins and 3) siderophores produced by bacteria and then secreted into the environment that scavenge iron, using their high affinity receptors. The siderophore-iron complexes are then collected by the bacteria. Most often, this tug-of-war is not won decisively by either side, but a given bacteria's ability to work-around the body's defenses determines the ability to live commensually within the body. BV is an example of a significant and most often long term invasion of bacteria that are able to live within the body's ecosystem while at the same time cause significant symptoms and risks.

Conceptually, nutritional immunity is designed to starve bacteria of their critical nutrients. Enrichment culture in the laboratory, on the other hand aims to do the opposite. It enhances the environment for a given bacterial species, taking into account its specific nutritional needs. In a biological system, these two concepts may coexist and may be individually manipulated to enhance selective bacterial growth of one species over another. However, being able to do so in vivo depends on 1) there being significant enough differences in nutritional needs of the respective species 2) a precise understanding of existing nutritional conditions and 3) an ability to modify and maintain control over the environment until the new environment and resulting microbiome has developed and is intrinsically stable. Our proposed treatment algorithm should meet these criteria in BV.

In an embodiment a new therapy may suppress pathological BV bacteria and L. iners, a transitional bacteria. In an embodiment the therapy restores beneficial bacteria associated with a normal microbiome. In another embodiment the therapy can be utilized as a follow up therapy, for example after a course of antibiotics to make the microbiome healthier and diminish the high recurrence of BV.

The invention is drawn to treatment of BV by iron sequestration and by vaginal supplementation of essential minerals and carbohydrates to encourage the growth of beneficial lactobacilli to the detriment of BV bacteria and Lactobacillus iners. In an embodiment a method of treating and preventing bacterial vaginosis recurrence in women comprising vaginally administering a therapeutic composition capable of sequestering iron and selectively reducing growth of bacteria associated with bacterial vaginosis.

In another embodiment the therapy can be used in conjunction with standard of care therapy, either before, during or after antibiotic treatment to help restore a more normal microbiome. Those therapeutic approaches that leave L. iners in place after the acute therapy for BV are likely to fail in the long term since when L. iners significantly persists, it provides an opportunity for BV bacteria to enter the vaginal microbiome and flourish.

Probiotics to replace L. iners with other Lactobacillus species is another interesting theory that has undergone much study in recent years with mixed results. The probiotic approach is thought to encourage growth or seed good bacteria but it does not complete the treatment algorithm proposed of changing the environment such that transitional bacterial like L. iners are at a disadvantage. This may explain the limited effectiveness of probiotics. Our understanding and approach, if combined with probiotic therapy, may significant increase the effectiveness of such therapy.

Beneficial lactobacilli require high manganese levels to grow optimally as shown by Macleod (1947). These have evolved in a highly unusual way to use manganese instead of iron for redox reactions. Like iron, manganese can transition between its Mn 2+ and Mn 3+/4+ states and provides the mechanism for electron transport. As can be expected for these non-iron-dependent bacteria, a much higher nutritional level of manganese is required if lactobacilli are to thrive. Lactobacillus selective (MRS) media designed by DeMan Rogossa and Sharpe (1960) have high levels of manganese and low levels of iron relative to other, more standard bacterial culture media. This specialized media permit lactobacilli to grow, yet do not provide sufficient nutrition for other bacteria to grow. This illustrates that at least in vitro, nutritional methods in a controlled environment can enhance the specific growth of lactobacilli over other bacteria that have sufficiently different metabolic and nutrient needs.

Falsen (1999) was the first to describe L. iners; over 100 years after vaginal lactobacilli were described by Doderlin. Unlike other lactobacilli, this unusual member of the species has an atypical microscopic appearance, has a requirement for iron, does not grow on MRS lactobacillus selective media, does not metabolize lactose (milk sugar) and produces L-lactate instead of D-lactate. It also does not require specific manganese supplementation in its growth media. These characteristics were unusual and noteworthy for a lactobacillus but are essentially normal for most if not all other BV bacteria. L. iners can source its iron requirements either directly or in symbiosis with BV bacteria such as G. vaginalis that lyse red blood cells during menses.

Iron is bound by human lactoferrin but some bacteria, including G. vaginalis, have developed specialized bacterial binding proteins that function to extract iron directly from human lactoferrin. This binding receptor is species specific and thus the binding protein that extracts iron from human lactoferrin and cannot function to extract iron from another species, for example bovine lactoferrin. We have confirmed G. vaginalis growth suppression with iron-containing bovine lactoferrin preparation. While some vaginal bacteria have been studied extensively, most of the multi-microbial BV bacteria have not. In an embodiment, a method and therapeutic composition are provided that deprives bacteria associated with BV of essential nutrients examples of such bacteria include L. iners, G. vaginalis, A. vaginae, M. curtisii, M. muleris, Megaspheraera spp and P. bivia.

A treatment algorithm has been developed that accomplishes this, by combining data with an in-depth knowledge of the genetic, metabolic, nutritional, and evolutionary aspects of the critical bacterial species in both the normal and BV microbiota. The algorithm capitalizes on the highly divergent metabolic and nutritional needs of the respective bacterial groups within the context of knowledge of the existing vaginal environment. It is theorized that once the vaginal environment is enhanced and a normal microbiome is restored, the microbiome will be robust and self-sustaining, as it is in most women without BV. Reinfection may recur but it will be less frequent if the microbiome is fully restored to normal with an abundance of functioning protective lactobacilli. Our theory has been developed and augmented by our data collection. We have developed specialized techniques to collect and analyze tiny amounts of pure vaginal fluid. We collected multiple specimens of vaginal fluid from normal woman and those with BV in order to understand the normal vaginal ecosystem and microbiome as well as the disease state, identifying and quantifying important parameters for each of them. We also determined baseline levels of iron, manganese, zinc and the protein calprotectin in vaginal fluid (a protein which binds managanese and zinc). A treatment plan is developed based on assembling the data points into a unified understanding and novel approach.

This original data shows that manganese levels are unexpectedly very low in the vaginal fluid of both normal women and those with BV. Manganese concentration in vaginal fluid is approximately 1 percent of the manganese concentration in optimized lactobacillus culture (MRS) media. Our assessment of this deficiency is based on the empiric, required levels of manganese to optimize in-vitro laboratory growth. The measured manganese levels in vaginal fluid are simply incompatible for robust growth of lactobacilli alone and certainly not in an environment of competitive, multi-species bacterial growth-as is seen is BV, that are dependent on iron and not dependent on manganese. Furthermore, these already low levels of manganese in vaginal fluid are additionally compromised since they are bound tightly by calprotectin, which are found to be greatly elevated in BV. On average, calprotectin is elevated 10 times relative to normal patients without BV. Calculations show that the elevated calprotectin levels in vaginal fluid seen in BV are sufficient to completely bind all of the manganese contained in the fluid. Thus manganese supplementation may be critical in treating BV and restoring of the normal vaginal microbiome.

Zinc levels in vaginal fluid of BV have also been measured. Recommended zinc levels for bacterial growth are not readily available as zinc toxicity is low and the capacity of bacteria to store zinc is high, thus contributing to a very wide range of acceptable zinc levels that will support bacterial growth. Analysis of the levels of calprotectin, its binding capacity and the levels of manganese and zinc show that in BV, calprotectin is sufficient to bind all available zinc and manganese contained in vaginal fluid. Furthermore, in an L. iners based microbiome that has not progressed to full blown BV, a significant increase in calprotectin is also seen. Thus, when an altered microbiome takes hold, the body appears to be an active conspirator in discouraging L. Crispatus-type re-growth. It does so by binding manganese and zinc, which as described above, affects L. Crispatus-type bacteria to a much greater extent than BV bacteria, and transitional bacteria like L. Iners. To determine the dose needed to obtain a given intravaginal concentration of a given therapeutic, we performed extensive pharmacokinetic studies on lactoferrin and an inert undigested marker (mannitol.) This data has established both concentration and duration of a given vaginal dose and the route of elimination.

This knowledge has been used to propose therapeutic doses for each compound, in order to achieve a final concentration of a given component based on calculations to attain the appropriate levels of these compounds in vaginal fluid. This is based on our pharmacokinetic data of vaginal tablet dissolution obtained in phase I studies, coupled with data of typical levels contained is serum and bacterial growth media.

Data shows that up to approximately 20% of a vaginal dose of bovine lactoferrin is broken down via vaginal or bacterial proteases. The remainder is expelled. This means that free iron is released from a dose of bovine lactoferrin that is partially saturated with iron and may become accessible to bacteria. Even if the released iron is immediately bound by the remaining lactoferrin, this proteolysis will inevitably increase the overall saturation percentage of the remaining bovine lactoferrin. In turn, it will also increase the saturation of human lactoferrin, since bovine and human forms exist in relative equilibrium considering that their bindings constants are similar. It also follows, that lowering the initial iron saturation of bovine lactoferrin will lower human lactoferrin's iron saturation levels and thus provide the least access to the bacterial binding proteins that scavenge iron from human lactoferrin. Since bovine lactoferrin is provided in great excess, lowering its initial saturation from 15% to 1% will take human lactoferrin with an estimated initial 30% saturation and reduce it to 1%, instead of to just 15% of the bovine lactoferrin was not further depleted. As compared to 15% bovine lactoferrin which would reduce human lactoferrin (saturated to 30 percent) by two fold, highly depleting the iron in bovine lactoferrin to 1% would result in a fold decrease in iron availability to the bacteria from human lactoferrin. Furthermore, others have shown an exponential decrease in siderophore scavenging effectiveness as iron saturation of transferrin decreases. Combining these data points indicate a potentially greatly increased clinical effectiveness, when the lactoferrin preparation is depleted to approximately 1% iron saturation. This is a key improvement over all others who have only utilized lactoferrin without iron depletion against BV and never postulated the utility and theory of depleting the iron from bovine lactoferrin to very low levels. Without an understanding of the ultimate competitive interation with human lactoferrin and the species-specific nature of binding proteins, coupled with the nutritional immunity approach, a 15% saturated bovine lactoferrin preparation is simply 15% less effective than a 1% preparation. This could easily be overcome by increasing the dose by 15%. As we have shown, this is not the case.

In summary, the aforementioned data illustrates several keys concepts: 1) L. iners is quite different from L. crispatus type bacteria in its metabolic and nutritional needs and is most similar to BV bacteria except in its antibiotic sensitivity, 2) in the vaginal environment, the body's nutritional immunity mechanism is quite active especially in BV with elevated inflammatory markers, lactoferrin levels and highly elevated calprotectin levels, and 3) the data provides strong clues and a consistent theory as to why L. iners and BV bacteria can out-compete L. crispatus, from a nutritional standpoint, and 4) provides insight to possible treatment algorithms. The details of the nutritional needs of BV bacteria and L. iners are highly significant if we are to modify the vaginal environment in line with specific nutritional parameters. Once established after displacing the normal vaginal microbiome, L. iners and other pathological bacteria are sustained as a viable microbiome since 1) they are able to source iron during menses and have highly developed mechanisms to source iron during other periods of the monthly cycle 2) they are able to source their requirements for trace amounts of Mn even from the low levels present in vaginal fluid, and 3) they can obtain sufficient levels of zinc with the aid of zinc acquisition pathways and 4) as a group, they are able to metabolize proteins in addition to glucose, and 5) BV bacteria and L. iners can be part of the same microbiome and share any of the above resources. One therapeutic nutritional impact approach may be to strengthen the body's attempt at nutritional immunity via human lactoferrin, by augmenting it with an excess of iron depleted bovine lactoferrin and lipocalin-2 (LCN2) against siderophores. Siderophores are compounds that are created by bacteria to bind iron, they are secreted into the environment, and reabsorbed after they have bound iron. They have an extremely high affinity for iron and can source iron even from lactoferrin. G. vaginalis has been shown to make these compounds under conditions of iron starvation. To counter bacterial siderophores, and as part of the cat-and-mouse struggles for iron, the body manufactures “anti-siderophore” compounds that bind siderophores, even those loaded with iron and thus prevent uptake by bacteria. The protein described is lipocalin2 (LCN2), a 25 kDa compound that has specific high affinity for the “catecholate-type” siderophores that G. vaginalis produces. In contradistinction, L. crispatus-type bacteria are relatively disadvantaged in the identical environment since 1) their manganese requirements are very significant and their requirements are difficult to source in the manganese-poor vaginal environment, 2) they all lack a zinc transport system to extract zinc from the low-zinc vaginal environment, and 3) they can only metabolize glycogen that has been predigested by a vaginal amylase enzyme that they do not even themselves possess. 4) the relatively easy availability of iron during menses is of no benefit to them since they do not use iron in their metabolic reactions or enzymes. Their nutrition may be positively augmented by providing for example, manganese, zinc and lactose.

The vaginal microbiome begins development before puberty and prior to significant competition from bacteria who can source iron from menses. We hypothesize that over time, a mature and stable L. crispatus-like microbiome is formed with adequate accumulated stores of manganese and zinc. Once established, well-recognized metabolic products of low-pH lactate and hydrogen peroxide provide a barrier to the colonization of other bacteria. If, however, a significant colonization with L. iners occurs, it will be entrenched in a vaginal environment that is well suited for its continued viability and has little reason to yield to recolonization by L. crispatus-type bacteria. This is especially true with the added deleterious effect of inflammation in BV where the body actively sequesters, via elevated calprotectin, the critical nutrients of manganese and zinc, that impact L. crispatus-like bacteria to a greater extent than L. iners. The precise levels of manganese, zinc and calprotectin identified in the vaginal fluid of normal and disease states, suggests a mechanism for BV and recurrence that has not been previously described. It attributes to and defines the underlying fundamental characteristics of the of a vaginal ecosystem which make it best suited at that point in time for the growth of transitional and BV microbiomes to the disadvantage of L. crispatus-type bacteria. As such, the treatment algorithm presented herein aims to change the vaginal environment in as many ways as possible to limit or reverse this preference.

In one embodiment our algorithm is focused on iron, manganese and zinc individually and in combination; precisely the minerals that body attempts most to sequester. Supplementing manganese at levels that overwhelm the body's ability to sequester manganese via calprotectin and to levels that are similar in magnitude to what has been found to work best in laboratory selective media for Lactobacillus species. As such, any advantage that iron-based bacteria have in the plentiful vaginal environment over those that use manganese. Zinc is augmented to remove the advantage that some BV bacteria have, including L. iners, by their ability to source critically necessary zinc in zinc-poor environments, such as the vagina. Zinc is also added to fill calprotectin's binding sites as the same sites that bind manganese also bind zinc.

Enhanced, iron-depleted bovine lactoferrin causes extreme iron starvation between menses for L. iners and other iron-dependent bacteria such as G. vaginalis, A. vaginae, Mobiluncus, etc. This preparation also significantly reduces the iron bound to human lactoferrin and makes iron unavailable to bacterial proteins that can only extract iron specifically from human lactoferrin.

Additionally an anti-siderophore compound, LCN2, to counteract the common bacterial siderophore mechanism works around lactoferrin's iron sequestration. As an additional incentive to encourage L. crispatus-like vs. L. iners/bv bacterial growth, lactose, a carbohydrate source for growth, that only L. crispatus-like bacteria can utilize can also be provided. There are other sugars such are sucrose that can only be metabolized by L. crisptus like bacteria. Any of these may accomplish a similar effect as long as they cannot be metabolized by L. iners.

Each component of the unified nutritional approach works independently via specific metabolic pathways and is innovative. Each component would be expected to independently modify the competitive vaginal environment in a positive way and may be efficacious to some extent. As an additive, each component can certainly be envisioned to increase the potency of competitive therapies. Combination of all components may cause even greater potency. In some cases titration or even removal of a given component may be necessary should there be the unintended consequence of growth of undesirable bacteria that also benefits from this vaginal environmental modification.

This encompassing theory and approach to treatment has never been proposed for BV, despite significant and ongoing efforts to control this disease since bacterial vaginosis was first described by Gardner in 1955. In this regard it is clearly not obvious even to those highly skilled in the art of gynecological medicine and pharmaceutical development.

The treatment methodology is designed to enhance only the growth of a single or multiple species of beneficial vaginal Lactobacilli. In some cases, these beneficial Lactobacilli are already present in the vagina in small numbers relative to the established L. iners-based microbiome, and thus only require nurturing to potentially predominate. In cases where an existing beneficial lactobacillus bacterium is not found or it is found desirable to “seed” the vagina with a specific species of probiotics, some or all of our described components may be combined as a therapeutic additive or course of treatment with such probiotic bacteria to better sustain their growth. Under these circumstances the novel vaginal therapy may be critical to enable or enhance such probiotic growth. The regimen may also be used in conjunction with or instead of antibiotics to treat BV or as an adjunctive therapy after BV is treated with antibiotics to further stabilize the microbiome and decrease recurrence. The precise utility of any novel medication or therapeutic approach can only be refined over time via extensive randomized controlled studies.

In brief, the levels of supplementation for manganese and zinc are set to exceed the highest possible level of calprotectin sequestration based on our original collected data regarding calprotectin elevation in vaginal fluid and the levels that are typical media concentrations that have been developed to grow these bacteria optimally in-vitro. These limits should not be viewed as limiting as during further study in larger cohorts, since these values may vary especially in biological systems where other environmental factors may impact a bacteria's access to the dosed trace metals.

For iron-depleted-lactoferrin, an excess is given to enhance competitive binding vis-a-vis human lactoferrin to make it unavailable for bacteria despite their binding proteins. Furthermore, the dose of bovine lactoferrin has been shown to decrease inflammation, a desirable goal if calprotectin and other inflammatory compounds are to be reduced. For LCN2, two orders of magnitude greater concentration than known human serum levels of LCN2 since the preparation is dosed topically on the vaginal surfaces with high bacterial counts. For lactose, the dosage used is similar to existing glucose and glycogen levels in vaginal fluid.

In an embodiment a method of treating and preventing bacterial vaginosis in women comprising vaginally administering a therapeutic composition capable of sequestering iron and selectively reducing growth of bacteria associated with bacterial vaginosis.

In an embodiment the therapeutic composition selectively deprives bacteria associated with bacterial vaginosis of essential nutrients.

Bacterial deprived of nutrients may be any bacteria desired to be inhibited or reduced. In an embodiment the bacteria deprived of nutrients may be selected from the group including, but not limited to, L. iners, G. vaginalis, A. vaginae, M. curtisii, M. mulieris, Megasperaeha spp, Prevotella and P. bivia.

In an embodiment the bacteria selected for nutrient deprivation is L. iners.

In an embodiment, the therapeutic composition enhances growth of beneficial bacteria, restoring the normal vaginal microbiome.

The beneficial bacteria to be promoted or increased may be any bacteria identified to be beneficial to the particular situation, often Lactobacillus bacteria other than L. iners. In an embodiment, the beneficial bacteria may be lactobacillus selected from the group including, but not limited to, L. crispatus, L. jensenii, L. gasseri, L. Helveticus, L. rhamnosus, L. reuteri, and L. casei.

In an embodiment the treatment decreases recurrence of bacterial vaginosis.

In an embodiment, the therapeutic composition is anti-inflammatory.

In an embodiment the therapeutic composition comprises at least one of or any combinations thereof selected from the group consisting of bovine lactoferrin, apolactoferrin, manganese, zinc, copper, lactose, lipocalin-2 and probiotics.

In an embodiment the therapeutic composition is formulated as a vaginal suppository.

In an embodiment further comprising administering the therapeutic composition before, during or following a course of antibiotics.

In an embodiment a method of seeding the vaginal microbiome comprising administering a therapeutic composition having selective compounds to enhance growth of beneficial lactobacillus species.

In an embodiment wherein seeding can be used as a treatment for bacterial vaginosis.

In an embodiment wherein seeding can be performed during or following antibiotic treatment for bacterial vaginosis.

In an embodiment a method of treating and preventing bacterial vaginosis in women comprising vaginally administering a therapeutic composition capable of sequestering iron, selectively reducing growth of bacteria associated with bacterial vaginosis and enhances growth of beneficial bacteria.

In an embodiment the treatment composition restores the vaginal microbiome.

A composition for the treatment of bacteria vaginosis comprising at least one of or any combinations thereof selected from the group consisting of bovine lactoferrin, apolactoferrin, manganese, zinc, copper, lipocalin-2, sugars and probiotics.

In an embodiment, wherein the composition sequesters iron, selectively reducing growth of bacteria associated with bacterial vaginosis and enhances growth of beneficial bacteria, restoring the normal vaginal microbiome.

The invention is drawn to improved compositions and methods thereof for vaginal drug delivery, including compositions for the treatment of bacterial vaginosis. In one embodiment of the invention, the composition comprises at least one active pharmaceutical ingredient, as formulated in the therapeutic composition, and delivered intravaginally presents a therapeutic effect in human hosts, either alone or in combination with other materials.

Nonlimiting examples of pharmaceutical ingredients useful in the present invention include proteins, peptides, minerals, trace elements, antibiotics, antifungals, anti-inflammatories, cytokines, hormones, anesthetics, analgesics, probiotics and fragments thereof. Preferred active pharmaceutical ingredients of the present invention for the treatment of bacterial vaginosis are selected from the group consisting of apolactoferrin, lactoferrin, manganese, lipocalin-2, zinc and lactose or combinations thereof. The amount of active pharmaceutical ingredient for the treatment of vaginosis in the therapeutic composition varies depending on the nature and potency of the therapeutic.

Optionally, the composition can also comprise a second active pharmaceutical ingredient selected from the group consisting of proteins, peptides, minerals, trace elements, antifungals, antibiotics, anti-inflammatories, cytokines, hormones, anesthetics, analgesics, probiotics and fragments thereof in addition to the vaginosis agent(s). The introduction of a second active pharmaceutical ingredient can result in an additive and/or synergistic therapeutic effect when combined with the first active pharmaceutical ingredient of the composition. In a preferred embodiment the second pharmaceutical ingredient is selected from the group consisting of one or more proteins (e.g. lactoferrin, apolactoferrin, lipocalin-2), trace elements (e.g. manganese, zinc, copper), sugars or a combination thereof.

In a further embodiment, the therapeutic composition of the present invention can be administered before, concomitantly or following administration of an antibiotic. In the case of bacterial vaginosis standard of care treatment is a course of antibiotics thus it is envisioned that the therapeutic composition could be administered in support of this therapy, being additive or synergetic and possibly improve efficacy of the antibiotic treatment, restoring the vaginal microbiome by supporting growth of beneficial lactobacillus. Non limiting examples of antibiotics include amoxicillin, ampicillin, ceftiofur, metronidazole, secnidazole, tinidazole, lincosamides, and clindamycin.

The therapeutic composition is delivered intravaginally for the treatment of bacterial vaginosis. By treatment of bacterial vaginosis, it is meant that the therapeutic composition is effective to prevent or reduce the incidence, severity and/or duration of the infection and symptoms associated with it. The therapeutic composition may also prevent the recurrence of infection. The therapeutic composition may selectively sequester iron selectively reducing growth of bacteria associated with bacterial vaginosis. The therapeutic composition may enhance growth of beneficial bacteria. The therapeutic composition can be in any convenient physical dosage form but is preferably in the form of a tablet of pessary. For example, when treating bacterial vaginosis, the therapeutic composition is preferably in the form of a tablet which can be introduced intravaginally.

In an embodiment the active pharmaceutical ingredient of the therapeutic composition is lactoferrin, and more preferably bovine lactoferrin. Lactoferrin is a complex 80 kD protein contained in the milk of all mammals and is also secreted from specialized granules in white blood cells and produced on mucosal surfaces. It is a significant component of the body's innate immune system, especially on mucosal surfaces and it is often elevated in response to infection. It is known to have anti-inflammatory properties. There is a slight to moderate variation of the protein structure across mammalian species, human and bovine lactoferrin are only 70% homogeneous in their amino acid sequence. As we will describe this provides a benefit over using human lactoferrin as an active pharmaceutical ingredient.

Lactoferrin is similar in structure and function to transferrin, the main protein in the blood that transports iron in the body. Lactoferrin or fragments thereof are envisioned to be used in this invention as a treatment for bacterial vaginosis. Concentrations of human lactoferrin are elevated in bacterial vaginosis as a natural consequence of the innate immune system response to infection. Gardnerella vaginalis, a predominate bacteria in bacterial vaginosis expresses lactoferrin binding proteins that bind human lactoferrin enabling the bacteria to extract iron from the human lactoferrin, counteracting its antimicrobial effect and instead exploiting human lactoferrin as a potential nutrient source. The lactoferrin binding proteins are species-specific, binding to human lactoferrin not bovine lactoferrin. Thus, while bovine lactoferrin will sequester iron away from Gardnerella vaginalis, human lactoferrin may serve as a source of iron for Gardnerella vaginalis. The presence of bovine lactoferrin, especially in high concentrations relative to the amounts of native human lactoferrin contained in vaginal fluid, will remove iron from human lactoferrin and decrease its iron saturation level. This is because human and bovine forms of lactoferrin have similar binding constants and can be expected to swap and share their iron amongst the total lactoferrin molecules; human or bovine.

Not to be bound by theory but to aid in the understanding of the invention, it is believed that the administration of lactoferrin, particularly bovine lactoferrin with its ability to bind and sequester iron with high affinity, will produce a microbiome modification in bacterial vaginosis (as iron is a key requirement for bacterial growth). By dosing lactoferrin, it is believed that the vaginal environment will become chronically iron-deficient and thereby less conducive to the growth of the pathogenic species seen in bacterial vaginosis that require iron. Regrowth of lactobacillus will be indirectly enhanced by less competition from other bacterial species that are suppressed. Lactobacillus, other than the transitional bacteria L. iners, themselves will not be directly impacted by this iron deprivation as they are one of only several bacterial species that do not require iron for growth, they instead utilize manganese, which lactoferrin binds relatively poorly.

The innovative approach uses bovine lactoferrin, to create a low-iron environment in the vagina in which healthy flora such as lactobacilli can thrive and where BV associated bacteria such as Gardnerella vaginalis are deprived of an essential nutrient, iron.

In another embodiment the therapeutic composition comprises manganese as an active pharmaceutical ingredient formulated in the therapeutic composition, for the treatment of bacterial vaginosis either alone or in combination with other materials. Manganese is a trace element and essential nutrient and can be administered as a therapeutic composition of the present invention to enhance the growth of lactobacillus dominated vaginal microbiome. In general, the lactobacillus family utilize manganese instead of iron and, therefore, their requirements for manganese are much higher than those of other species. Manganese supplementation to encourage the selective bacterial growth of Lactobacilli in patients with bacterial vaginosis, as a primary treatment or as an accessory treatment with for bacterial vaginosis at levels and duration sufficient to support robust growth of beneficial lactobacilli. This will enhance the ability of lactobacilli to compete with and ultimately displace those bacteria that are dependent on iron, as all BV associated bacteria are, including the transitional bacteria L. iners. As above, without intervention, these BV associated bacteria have a built-in advantage over beneficial lactobacilli since they can obtain iron during menses and many have otherwise developed iron binding receptor proteins or specialized iron scavenging proteins (siderophores) to satisfy their iron needs. We believe that a therapy that enhances the growth of L. crispatus type bacteria without similarly enhancing the growth the transitional bacterial L. iners is particularly desirable. Manganese supplementation will provide a necessary nutrient that is currently deficient in those that have developed BV, even after apparent clinical symptomatic recovery.

In another embodiment the therapeutic composition containing manganese contains a second active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing manganese can be administered following administration of another pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing manganese can be administered before, concomitantly or following administration of an antibiotic. In an embodiment the therapeutic composition containing manganese is administered following antibiotic treatment for bacterial vaginosis. The use of antibiotics is considered the standard of care treatment for bacterial vaginosis. Non limiting examples of antibiotics used include amoxicillin, ampicillin, ceftiofur, metronidazole, secnidazole, tinidazole, lincosamides, and clindamycin.

In another embodiment the therapeutic composition comprises zinc as an active pharmaceutical ingredient formulated in the therapeutic composition, for the treatment of bacterial vaginosis either alone or in combination with other materials.

In another embodiment the therapeutic composition containing zinc contains a second active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing zinc can be administered following administration of another pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing zinc can be administered before, concomitantly or following administration of an antibiotic. In an embodiment the therapeutic composition containing zinc is administered following antibiotic treatment for bacterial vaginosis. The use of antibiotics is considered the standard of care treatment for bacterial vaginosis. Non limiting examples of antibiotics used include amoxicillin, ampicillin, ceftiofur, metronidazole, secnidazole, tinidazole, lincosamides, and clindamycin.

In another embodiment the active pharmaceutical ingredient is apolactoferrin, a form of lactoferrin that contains little iron (approximately <2% of iron binding sites occupied). In another embodiment the therapeutic composition comprises apolactoferrin as an active pharmaceutical ingredient, a binder and a filler that, as formulated in the therapeutic composition, for the treatment of bacterial vaginosis either alone or in combination with other materials. The therapeutic composition delivered intravaginally, containing apolactoferrin in a therapeutic composition can be formulated as immediate release or a slow release composition.

In another embodiment the therapeutic composition containing apolactoferrin contains a second active pharmaceutical ingredient for example proteins, peptides, minerals, antibiotics, anti-inflammatories, cytokines, hormones, anesthetics, analgesics, probiotics and fragments thereof.

In another embodiment the therapeutic composition containing apolactoferrin can be administered concomitantly with a second active pharmaceutical ingredient for example proteins, peptides, minerals, antibiotics, anti-inflammatories, cytokines, hormones, anesthetics, analgesics, probiotics and fragments thereof.

In another embodiment the therapeutic composition containing apolactoferrin can be administered before or following administration of another active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing apolactoferrin can be administered before, concomitantly or following administration of an antibiotic. In an embodiment the therapeutic composition containing lactoferrin is administered following antibiotic treatment for bacterial vaginosis.

In another embodiment the therapeutic composition comprises lipocalin-2 as an active pharmaceutical ingredient formulated in the therapeutic composition, for the treatment of bacterial vaginosis either alone or in combination with other materials.

In another embodiment the therapeutic composition containing lipocalin-2 can be administered with an second active pharmaceutical ingredient for example proteins, peptides, minerals, antibiotics, anti-inflammatories, cytokines hormones, anesthetics, analgesics, probiotics and fragments thereof.

In a further embodiment, the therapeutic composition containing lipocalin-2 can be administered with another pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing lipocalin-2 can be administered before, concomitantly or following administration of an antibiotic. In an embodiment the therapeutic composition containing lipocalin-2 is administered following antibiotic treatment for bacterial vaginosis.

In a further embodiment, the therapeutic composition contains lactose as a second ingredient.

In another embodiment, a kit may comprise the therapeutic compositions described above. In a specific embodiment, the kit may comprise daily active dosage units comprising a pharmaceutical ingredient in a therapeutic composition, herein the therapeutic composition allows for daily dosing regimen as prescribed, and wherein the initial administration of the therapeutic composition establishes its effect on bacterial vaginosis.

In another embodiment a kit may comprise the therapeutic composition of the present invention as well as an antibiotic formulation that is standard of care, thus having the ability to provide both antibiotic and therapeutic composition for daily dosage regimen.

In another embodiment, the present disclosure provides a method for making the slow release composition comprising: combining one or more active pharmaceutical ingredients, a gelling agent, a binder and a filler, mixing the ingredients to combine, and pressing the ingredients into a tablet. Excipients can be added to improve the desired characteristics of the tablet.

The invention is drawn to improved compositions and methods thereof for vaginal drug delivery, including compositions for the treatment of bacterial vaginosis. In one embodiment of the invention, the therapeutic composition comprises at least one active pharmaceutical ingredient, a binder, a filler and a gelling agent that, as formulated in the therapeutic composition, and delivered intravaginally presents a therapeutic effect in human hosts, either alone or in combination with other materials.

In a preferred embodiment, the therapeutic composition comprises at least one active pharmaceutical ingredient, a binder a filler and a gelling agent that, as formulated in the therapeutic composition, presents a therapeutic effect in human hosts, for the treatment of bacterial vaginosis either alone or in combination with other materials.

In an embodiment the composition is a slow release composition for delivery of the therapeutic composition. In that regard, the therapeutic effect may be due to the direct action of the pharmaceutical ingredient of the composition or may be due to one or more other materials additively or synergistically interacting with the pharmaceutical ingredient. The present invention provides a therapeutic composition delivered intravaginally for vaginal drug delivery. An example of such a therapeutic is a therapeutic composition delivered intravaginally for the treatment of bacterial vaginosis. By treatment of bacterial vaginosis, it is meant that the therapeutic composition is effective to prevent or reduce the incidence, severity and/or duration of the infection. The therapeutic composition may also prevent reoccurrence of the infection.

Nonlimiting examples of pharmaceutical ingredients useful in the present invention include proteins, peptides, minerals, trace elements, antibiotics, antifungals, anti-inflammatories, cytokines, hormones, anesthetics, analgesics, probiotics and fragments thereof. Preferred active pharmaceutical ingredients of the present invention for the treatment of bacterial vaginosis are selected from the group consisting of lactoferrin, manganese, lipocalin-2 and apolactoferrin or combinations thereof. The amount of active pharmaceutical ingredient for the treatment of vaginosis in the therapeutic composition varies depending on the nature and potency of the therapeutic.

Optionally, the composition can also comprise a second active pharmaceutical ingredient selected from the group consisting of proteins, peptides, minerals, trace elements, antifungals, antibiotics, anti-inflammatories, cytokines, hormones, anesthetics, analgesics, probiotics and fragments thereof in addition to the vaginosis agent(s). The introduction of a second active pharmaceutical ingredient can result in an additive and/or synergistic therapeutic effect when combined with the first active pharmaceutical ingredient of the composition. In a preferred embodiment the second pharmaceutical ingredient is selected from the group consisting of one or more proteins (e.g. lactoferrin, apolactoferrin, lipocalin-2), trace elements (e.g. manganese) or a combination thereof. Typically, the amount of a second active pharmaceutical ingredient in the therapeutic composition is from about 0.01% by weight of the composition to about 20% weight of the composition.

The therapeutic composition of the present invention includes a binder, used to hold the ingredients together. They also give weight and allow small active ingredients to be combined into a capsule or tablet. Generally, binders are typically sugar derivatives and include: lactose, sucrose, mannitol, microcrystalline cellulose, malitol, sorbitol, xylitol etc. However, they can also include water-soluble hydroxyalkyl celluloses such as polyvinylpyrrolidone, povidone, xantham gum, celluloses (hydroxypropylmethylcellulose, hydroxypropyl cellulose) gelatin, starch, polyethylene glycol or any combination thereof. It is important to point out that there are some compounds which can be used both as binders and bioadhesive gelling agents for example polymers such as polyvinylpyrrolidone and poloxamers. Typically, when included in the therapeutic composition, the amount of binder in the therapeutic composition is from about 0.5% by weight of the composition to about 10% weight of the composition.

The therapeutic composition of the present invention includes a filler. Fillers add bulk to products making small active components easier for administration. Fillers also disperse large active components such as proteins, facilitating uniform slow release. Non-limiting examples include: lactose, sucrose, mannitol, magnesium stearate, glucose, plant cellulose, calcium carbonate etc. It is important to point out that there are some compounds which can be used as both binders and fillers such as sugars. Typically, when included in the therapeutic composition, the amount of filler in the therapeutic composition is from about 20% by weight of the composition to about 80% weight of the composition.

The therapeutic composition of the present invention includes a gelling agent, and preferably one which is bioadhesive. Common gelling agents include acacia, alginic acid, bentonite, carbomers, carboxymethyl cellulose, ethylcellulose, gelatin, hydroxyethylcellulose, hydroxypropylcellulose, magnesium aluminum silicate, methylcellulose, poloxamers, polyvinyl alcohol, sodium alginate, tragacanth, and xanthan gum. Sane active pharmaceutical ingredients during the manufacture process take on the properties of gelling agents. Non-limiting examples of such active pharmaceutical ingredients of the invention include lactoferrin and proteins. As bioadhesive gelling agents are preferred nonlimiting examples more specifically include poloxamer 407, poloxamer 188, polyvinylpyrrolidone, celluloses, chitosans, mucin glycoproteins, trefoil peptides and polycarbophils. Furthermore, more than one of these exemplary bioadhesive polymers may be included in the composition to provide the desired characteristics. Particularly preferred bioadhesive polymers include celluloses, polyvinylpyrrolidone and poloxamer 407. Typically, when included in the therapeutic composition, the amount of bioadhesive in the therapeutic composition is from about 0.5% by weight of the composition to about 10% weight of the composition.

The therapeutic composition of the present invention may further comprise one or more excipients, including lubricants, glidants and combinations thereof. The therapeutic composition of the present invention may include a lubricant, examples include magnesium stearate, stearic acid, or glyceryl dibehenate which aid in manufacturing. When included in the therapeutic composition, the amount of lubricant in the therapeutic composition is from about 0.1% by weight of the composition to about 5% weight of the composition.

The therapeutic composition of the present invention may include a glidant, examples include magnesium stearate, silicon dioxide, starch. Glidant will be added when flowability is required in the composition. The composition can also comprise other components to the extent that the presence of said components is not inconsistent with performance objectives of the composition. When included in the therapeutic composition, the amount of glidant in the therapeutic composition is from about 0.1% by weight of the composition to about 4% weight of the composition.

In a preferred embodiment the components of the therapeutic composition comprising at least one active pharmaceutical ingredient, binder, lubricant, filler, glidant and a bioadhesive polymer as formulated in the therapeutic composition, presents a therapeutic effect in human hosts, for the treatment of bacterial vaginosis either alone or in combination with other materials.

In a further embodiment, the therapeutic composition of the present invention can be administered before, concomitantly or following administration of an antibiotic. In the case of bacterial vaginosis standard of care treatment is a course of antibiotics thus it is envisioned that the therapeutic composition could be administered in support of this therapy, being additive or synergetic and possibly improve efficacy of the antibiotic treatment. Non limiting examples of antibiotics include amoxicillin, ampicillin, ceftiofur, metronidazole, secnidazole, tinidazole, lincosamides, and clindamycin.

The therapeutic composition is delivered intravaginally for the treatment of bacterial vaginosis. By treatment of bacterial vaginosis, it is meant that the therapeutic composition is effective to prevent or reduce the incidence, severity and/or duration of the infection and symptoms associated with it. The therapeutic composition may also prevent the recurrence of infection. The therapeutic composition can be in any convenient physical dosage form but is preferably in the form of a tablet of pessary. For example, when treating bacterial vaginosis, the therapeutic composition is preferably in the form of a tablet which can be introduced intravaginally. The components of the therapeutic composition are combined and compressed at high forces to form a tablet. It was observed that upon contact with the low moisture environment of the vagina the outer layer of the tablet was observed to transition to a gel, due to the hygroscopic and gelling nature of its chosen excipients as it contacts moisture in the vagina. This gel forms a coating that surrounds the tablet and acts to prevent further fluid contact and tablet dissolution. Further dissolution will only occur when, over time, the gel is naturally dispersed onto the vaginal mucosa away from the tablet. Once this occurs, the therapeutic composition is slowly released and further prolongs the contact time of the tablet to the vaginal mucosa. To our knowledge no one had yet exploited the limited fluid environment with novel compositions that slowly wet and shed drug rather than disintegrating and releasing drug more quickly. This is in contrast to compositions that include disintegrant, which burst the tablet, increase the exposed surface area of the composition, and enhance dissolution rates. Intravaginal drug delivery by this slow release mechanism has not been previously described and may be applicable to a variety of drugs for intravaginal delivery.

In another embodiment the tablet dissolution and rate of release of active ingredients can be further modified by making bilayer tablets. The dissolution and release rate of a formulation containing an active pharmaceutical ingredient can be prolonged by covering a surface or side of the tablet with other components such as active pharmaceutical ingredients, fillers or gelling agents. For example, a bilayer tablet containing an “A” layer or side of lactoferrin and a “B” layer or side of lactose, manganese and gelling agent is predicted to release lactoferrin more slowly than a single layer tablet containing the same amount of lactoferrin as the surface area of lactoferrin accessible to water is decreased by the B layer or side. The B layer of the formulation will additionally deliver lactose and manganese to the vaginal environment. The rate of release of active such as manganese from the B side of a bilayer tablet can also be modulated.

In another embodiment, a tablet may consist of two or more distinct layers, with each layer having its own blend of excipients and therapeutics and each having its own phamaco-kinetic dissolution profile depending on the dissolution needs of the particular therapeutic.

In another embodiment, a tablet may consist of two or more distinct sides, with each side having its own blend of excipients and therapeutics and each having its own phamaco-kinetic dissolution profile depending on the dissolution needs of the particular therapeutic.

In some embodiments, a vaginal suppository tablet may be formulated for sustained release with particular dissolution characteristics according to identified dissolution test protocols. In one embodiment, a vaginal suppository tablet formulated for sustained release is formulated to dissolve all of a target component or components (e.g., manganese, lactoferrin, lipocalin-2, zinc, etc.) from the tablet over a time period of from 20 to 90 minutes, and preferably over a time of from 30 to 90 minutes, according to a dissolution test procedure (Test Protocol A) of U.S. Pharmacopeia (USP) <711> into 900 mL of dissolution medium of 0.2M acetate buffered solution at pH 4.5±0.05 and 37±0.5° C. in a basket dissolution apparatus with 1 liter vessel volume and mixing speed of 75 revolutions per minute, with 5 mL syringe samples of dissolution medium collected at intervals of 15, 30, 45, 60, 90 and 120 minutes and analyzed to determine extent of dissolution of target component. The tablet is placed in the wire basket of the basket apparatus, using a sinker as appropriate, and submerged in the dissolution medium mixed at the noted rotational speed Determination of extent of dissolution of target component (e.g., manganese) may include analysis of collected samples to determine the dissolve concentration of the target component and calculation of degree of dissolution of the target component by difference between the total starting amount of the target component in the vaginal suppository tablet and the total amount of the component dissolved into the dissolution medium. Concentration of the target component in analyzed samples may use techniques appropriate for the particular target component (e.g., appropriate for concentration analysis of manganese, lactoferrin, lipocalin-2, zinc, etc.). One analytical technique may be unitraviolet-visible light spectrophotometry at 280 nm for analysis of proteinaceous components such as lactoferrin and lipocalin-2, and analytical readings may be compared to a standard curve of for the target component of known concentrations. The complete dissolution of the target component or components may or may not correspond to complete dissolution of the tablet, but preferably the completed dissolution of the target component does correspond to complete dissolution of the tablet.

In another embodiment, a vaginal suppository tablet formulated for sustained release, is formulated to dissolve all of a target component or components (e.g., manganese, lactoferrin, lipocalin-2, zinc, etc.), and preferably to dissolve all of the tablet, over a period of time of from 2 hours to 10 hours, and preferably from 3 hours to 10 hours, according to the following test procedure (Test Protocol B):
a 2 milliliter sample of normal saline (0.9 gram per liter sodium chloride) as a dissolution solution at 37° C. is placed into a 5 milliliter tube;
a tablet is placed in the tube containing the dissolution solution and the tube is sealed and secured to a rotary shaker;
the rotary shaker with the secured tubes is operated at a 30 degree tilt, speed of turns per minute and temperature setting of 37° C.;
the shaker is operated for 10 hours, pausing briefly for intermediate visual observation of tube contents at 0.5, 1, 2, 4 and 8 hours; and
dissolution status of the tablet is record for each time point of 0.5, 1, 2, 4, 8 and 10 hours and the time point is recorded at which total tablet dissolution occurs or that total tablet dissolution did not occur during the 10 hour test period as determined by visual observation.

Test Protocol B is a low-volume test designed to be more representative of the local vaginal environment. The test using Test protocol B may continue beyond 10 hours if desired, for example to test compositions that do not fully dissolve during 10 hours. For example, the test may be extended to 24 hours duration, or any other desired duration, with test observations taken at additional time points beyond 10 hours. A sustained release vaginal suppository tablet may be formulated to satisfy the dissolution conditions of either one of Test Protocol A or Test Protocol B, or both Test Protocol A and Test Protocol B.

In some preferred embodiments, a vaginal suppository tablet provides sustained release of an active ingredient or ingredients in vivo after intravaginal placement, wherein all of the active ingredient or ingredients dissolve, and preferably the entire tablet dissolves, over a time period of not shorter than 12 hours, and preferably over a time period of 12 to 36 hours, and during such time period the tablet preferably completely dissolves. Each of Test Protocol A and Test Protocol B, individually and/or together, have been identified as useful for in vitro evaluation of performance of vaginal suppository compositions to provide the desired in vivo sustained release performance characteristics. As may be appreciated, a broad range of tablet formulations may be prepared to provide the desired sustained release characteristics. Formulation of vaginal suppository tablets with varying concentrations of active ingredients, gelling agents, binders, fillers, and other excipients and processing aids (e.g., lubricants and glidants) and tablet structure (e.g., bilayer characteristics) may be easily formulated and tested in vitro to identify particular tablet compositions and structures to provide desired in vitro sustained release characteristics, which can then be tested and confirmed for desired in vivo sustained release characteristics.

In an embodiment the active pharmaceutical ingredient of the therapeutic composition is lactoferrin, and more preferably bovine lactoferrin. Lactoferrin is a complex 80 kD protein contained in the milk of all mammals, is secreted from specialized granules in white blood cells and is also present on mucosal surfaces. It is a significant component of the body's innate immune system, especially on mucosal surfaces and it is often elevated in response to infection. There is a slight to moderate variation of the protein structure across mammalian species, human and bovine lactoferrin are only 70% homogeneous in their amino acid sequence. As we will describe this provides a benefit over using human lactoferrin as an active pharmaceutical ingredient.

Lactoferrin is similar in structure and function to transferrin, the main protein in the blood that transports iron in the body. An important difference between lactoferrin and transferrin is the pH at which they release iron. Transferrin releases iron at pH 5.5 and serves a transport role. Lactoferrin still binds iron in the low pH environment of the GI tract and vaginal mucosal where it acts as an iron scavenger and a selective antimicrobial agent.

Lactoferrin is often described as a multifunctional protein as it appears to perform many functions in the body. These include binding iron, an anti-inflammatory effect, and a growth stimulator of intestinal mucosa. Specific lactoferrin receptors have been discovered on the surface of cells in the proximal small bowel and on some bacteria. Lactoferrin or fragments thereof are envisioned to be used in this invention as a treatment for bacterial vaginosis. Concentrations of human lactoferrin are elevated in bacterial vaginosis as a natural consequence of the innate immune system response to infection. Gardnerella vaginalis, a predominate bacteria in bacterial vaginosis expresses lactoferrin binding proteins that bind human lactoferrin enabling the bacteria to extract iron from the human lactoferrin, counteracting its antimicrobial effect and instead exploiting human lactoferrin as a potential nutrient source. The lactoferrin binding proteins are species-specific, binding to human lactoferrin not bovine lactoferrin. Thus, while bovine lactoferrin will sequester iron away from Gardnerella vaginalis, human lactoferrin may serve as a source of iron for Gardnerella vaginalis. The presence of bovine lactoferrin, especially in high concentrations relative to the amounts of native human lactoferrin contained in vaginal fluid, will remove iron from human lactoferrin and decrease its iron saturation level. This is because human and bovine forms of lactoferrin have similar binding constants and can be expected to swap and share their iron amongst the total lactoferrin molecules; human or bovine.

Not to be bound by theory but to aid in the understanding of the invention, it is believed that the administration of lactoferrin, particularly bovine lactoferrin with its ability to bind and sequester iron with high affinity, will produce a microbiome modification in bacterial vaginosis (as iron is a key requirement for bacterial growth). By dosing lactoferrin once daily and achieving a sustained dose via the slow release therapeutic composition, it is believed that the vaginal environment will become chronically iron-deficient and thereby less conducive to the growth of the pathogenic species seen in bacterial vaginosis that require iron. Regrowth of lactobacillus will be indirectly enhanced by less competition from other bacterial species that are suppressed. Lactobacillus themselves will not be directly impacted by this iron deprivation as they are one of only several bacterial species that do not require iron for growth, they instead utilize manganese, which bovine lactoferrin binds relatively poorly. The delineation of the precise mechanism of action of lactoferrin in the vaginal environment and its impact on pharmaceutical development and the benefit of a slow release lactoferrin formulation has not been previously described. Indeed, it is counterintuitive, as typical antimicrobial pharmacokinetics aim to achieve a rapid tablet dissolution to obtain high peak levels and thus bacterial toxicity of the active pharmaceutical ingredient. Moreover, since human lactoferrin is elevated in vaginal fluid in bacterial vaginosis, the idea of using bovine lactoferrin or apolactoferrin which contains less than 1-2% iron is novel.

In bacterial vaginosis, sustained therapeutic levels of lactoferrin are highly beneficial since bacteria will be most significantly and adversely impacted by iron deprivation if it is sustained over the course of the expected multi day treatment. If iron deprivation is intermittent, bacteria will replenish their stores of iron during periods of available iron, and the treatment will not be effective. Stated differently, the same administered dose of lactoferrin may or may not be effective, depending on the precise formulation and the duration of lactoferrin's contact with the vaginal mucosa. This is unlike other topical or systemic antimicrobials that kill or suppress bacteria via toxicity, and where intermittent and peak levels may still be highly effective or even desirable.

Our innovative approach uses bovine lactoferrin to create a low-iron environment in the vagina in which healthy flora such as lactobacilli can thrive and where BV-associated bacteria such as Gardnerella vaginalis are deprived of an essential nutrient, iron. A slow-release formulation that maintains a continuously iron-deficient state is critical to the success of this nutritional immunity-based approach to the treatment of BV. Short-acting lactoferrin formulations may only suppress but not eliminate BV-associated bacteria and will likely encounter the same problems of high rates of recurrence seen with antibiotics. This slow release approach may also be applied to other drugs and indications in which vaginal delivery is appropriate.

In one embodiment the slow release therapeutic composition comprises lactoferrin, a binder, a filler and a gelling agent that, as formulated in the therapeutic composition, presents a therapeutic effect in human hosts, for the treatment of bacterial vaginosis either alone or in combination with other materials. In that regard, the therapeutic effect may be due to the direct action of the pharmaceutical ingredient of the composition or may be due to one or more other materials additively or synergistically interacting with the pharmaceutical ingredient. In a preferred embodiment the lactoferrin is bovine lactoferrin. The present invention provides a therapeutic composition delivered intravaginally for the treatment of bacterial vaginosis. By treatment of bacterial vaginosis, it is meant that the therapeutic composition is effective to prevent or reduce the incidence, severity and/or duration of the infection. The therapeutic composition may also prevent reoccurrence of the infection.

In a preferred embodiment the therapeutic composition comprises bovine lactoferrin, a bioadhesive gelling agent, a binder, lubricant, and filler. Typically, when included in the therapeutic composition, the amount of lactoferrin formulated in a tablet is from about 40% by weight of the composition to about 80% weight of the composition. The components of the therapeutic composition are combined and the lactoferrin is dispersed throughout the composition which is compressed under high forces to form a tablet. Properties of this compressed tablet include lactoferrin having gel like properties and slow dissolution when the composition is comes in contact with low moisture environment in the vagina, and that it dissolves slowly. Characteristic changes in the tablet following exposure to moisture include, formation of an outer gel in response to contact with moisture, as the outer gel forms, the inner portions of the tablet stay dry and hard, even if immersed in excess amounts of fluid. Only when the gel is successively dispersed and the tablet is all but dissolved, does the final inner portion of the tablet become wet, as it comes into contact with the dissolving solution. The tablet is uniform in composition. Thus, the outer gel layers and inner portions have the same composition. The slow wetting properties of the composition achieve slow release by gradually changing the surface area of the tablet. These distinct characteristics of our novel tablet contrast with other vaginal medicinal tablets including those containing lactoferrin that contain disintegrants, which cause the tablet to fracture and rapidly increase surface area or lack gelling or bioadhesive elements. An example is Difesan, which is an adjuvant treatment containing lactoferrin along with a disintegrant, crospovidone. Difesan is marketed in Italy for the treatment of pre-term birth, female infertility, cervical and vaginal infections. Its instructions for use that state that “the tablet should be introduced into the vagina, in depth, in order to promote rapid disintegration”. These types of compositions are designed for rapid dissolution and for high rather than sustained pharmaceutical levels. These rapidly dissolving tablets either rapidly wet completely, or most often fragment into multiple particles some which may be expelled by the vagina without ever dissolving. When this occurs, it decreases both the duration of pharmacological activity and also the average levels of the pharmaceutical agent.

In another embodiment the therapeutic composition containing lactoferrin contains a second active pharmaceutical ingredient for example proteins, peptides, minerals, antibiotics, anti-inflammatories, cytokines, hormones, anesthetics, analgesics, probiotics and fragments thereof.

In another embodiment the therapeutic composition containing lactoferrin can be administered with a second active pharmaceutical ingredient for example proteins, peptides, minerals, antibiotics, anti-inflammatories, cytokines, hormones, anesthetics, analgesics, probiotics and fragments thereof.

In another embodiment, the therapeutic composition containing lactoferrin can be administered before administration of another pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing lactoferrin can be administered following administration of another active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing lactoferrin can be administered concomitantly with administration of another active pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing lactoferrin can be administered before, concomitantly or following administration of an antibiotic. In an embodiment the therapeutic composition containing lactoferrin is administered following antibiotic treatment for bacterial vaginosis. The use of antibiotics is considered the standard of care treatment for bacterial vaginosis. Non limiting examples of antibiotics used include amoxicillin, ampicillin, ceftiofur, metronidazole, secnidazole, tinidazole, lincosamides, and clindamycin. However, over time antibiotic resistance can occur making the antibiotic therapy less effective. The high rate of recurrence in BV suggests that treatment with antibiotics is merely suppression therapy and that pockets of live bacteria remain within antibiotic-resistant biofilm. Treatment with lactoferrin, which kills bacteria via different mechanisms than traditional antibiotics and can disrupt biofilm, will potentially eliminate residual bacteria following antibiotic treatment. The therapeutic composition as envisioned could be administered in support of antibiotic therapy, being additive or synergetic and possibly improving efficacy of the antibiotic treatment.

In another embodiment the therapeutic composition comprises manganese as an active pharmaceutical ingredient, a binder and a filler that, as formulated in the therapeutic composition, for the treatment of bacterial vaginosis either alone or in combination with other materials. The therapeutic composition delivered intravaginally, containing manganese in a therapeutic composition can be formulated as immediate release or a slow release composition. In that regard, the therapeutic effect may be due to the direct action of the pharmaceutical ingredient of the composition or may be due to one or more other materials synergistically interacting with the pharmaceutical ingredient. The present invention provides a slow release therapeutic composition comprising manganese as an active pharmaceutical ingredient, a binder a filler and a gelling agent that, as formulated in the therapeutic composition delivered intravaginally for the treatment of bacterial vaginosis. By treatment of bacterial vaginosis, it is meant that the therapeutic composition is effective to prevent or reduce the incidence, severity and/or duration of the infection. The therapeutic composition may also prevent reoccurrence of the infection.

Manganese is a trace element and essential nutrient and can be administered concomitantly with a therapeutic composition of the present invention in which lactoferrin is the active pharmaceutical ingredient. In an embodiment manganese is added as a second active pharmaceutical ingredient to a slow release therapeutic composition containing lactoferrin, a binder, a filler and a gelling agent to additionally enhance the growth of lactobacillus dominated vaginal microbiome. This approach is based on utilizing the iron-sequestering capacity of lactoferrin to modify the vaginal microbiome and restore the normal resident microbiota. In newborns, lactoferrin from mother's milk promotes a predominantly-lactobacillus and bifidobacterial gut microbiome. As in the intestinal microbiota, lactobacilli also comprise a healthy vaginal microbiota. The iron-sequestration mechanism of lactoferrin action depends on the fact that virtually all bacteria need iron. Lactobacilli are an exception and are not suppressed because they do not require iron, using manganese instead. Our measurements of manganese levels in vaginal fluid show that they are low, relative to optimal levels for lactobacillus growth, further diminishing the availability of manganese to bacteria. Furthermore, our original research has identified significant levels of calprotectin in vaginal fluid, a specialized protein that is known to bind manganese with a high affinity and makes it unavailable for use by bacteria. The necessity of robust levels of available manganese is further highlight by the fact that lactobacillus selective media such as DeMan, Rogosa and Sharpe media, contain no supplemental iron but do contain an excess of manganese as compared to typical human physiological levels and other bacterial growth media. Manganese supplementation to encourage the selective bacterial growth of Lactobacilli in patients with bacterial vaginosis, as a primary treatment or as an accessory treatment with lactoferrin for bacterial vaginosis is a novel idea that has not been previously described.

In another embodiment the therapeutic composition containing manganese contains a second active pharmaceutical ingredient for example proteins, peptides, minerals, antibiotics, anti-inflammatories, cytokines, hormones, anesthetics, analgesics, probiotics and fragments thereof. In a preferred embodiment apolactoferrin is added as a second active pharmaceutical ingredient to a therapeutic composition containing manganese to additionally enhance the growth of a lactobacillus dominated vaginal microbiome.

In another embodiment a therapeutic composition containing manganese can be administered with a second active pharmaceutical ingredient for example proteins, peptides, minerals, antibiotics, anti-inflammatories, cytokines, hormones, anesthetics, analgesics, probiotics and fragments thereof. In a preferred embodiment the second active pharmaceutical ingredient is apolactoferrin.

In a further embodiment, the therapeutic composition containing manganese can be administered concomitantly with another pharmaceutical ingredient for example proteins, peptides, minerals, antibiotics, anti-inflammatories, cytokines, hormones, anesthetics, analgesics, probiotics and fragments thereof.

In another embodiment, the therapeutic composition containing manganese can be administered before administration of another pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing manganese can be administered following administration of another pharmaceutical ingredient for example proteins, peptides, minerals, antibiotics, anti-inflammatories, cytokines, hormones, anesthetics, analgesics, probiotics and fragments thereof.

In a further embodiment, the therapeutic composition containing manganese can be administered before, concomitantly or following administration of an antibiotic. In an embodiment the therapeutic composition containing manganese is administered following antibiotic treatment for bacterial vaginosis. The use of antibiotics is considered the standard of care treatment for bacterial vaginosis. Non limiting examples of antibiotics used include amoxicillin, ampicillin, ceftiofur, metronidazole, secnidazole, tinidazole, lincosamides, and clindamycin.

In another embodiment the therapeutic composition comprises lactoferrin, manganese, a gelling agent, binder, lubricant, glidant and filler that, as formulated in the therapeutic composition, presents a therapeutic effect in human hosts, for the treatment of bacterial vaginosis. In that regard, the therapeutic effect may be due to the direct action of the pharmaceutical ingredient of the composition or may be due to one or more other materials synergistically interacting with the pharmaceutical ingredient. The present invention provides a therapeutic composition delivered intravaginally for the treatment of bacterial vaginosis. By treatment of bacterial vaginosis, it is meant that the therapeutic composition is effective to prevent or reduce the incidence, severity and/or duration of the infection. The therapeutic composition may also prevent reoccurrence of the infection.

In another embodiment the active pharmaceutical ingredient is apolactoferrin. In another embodiment the therapeutic composition comprises apolactoferrin as an active pharmaceutical ingredient, a binder and a filler that, as formulated in the therapeutic composition, for the treatment of bacterial vaginosis either alone or in combination with other materials. The therapeutic composition delivered intravaginally, containing apolactoferrin in a therapeutic composition can be formulated as immediate release or a slow release composition. In that regard, the therapeutic effect may be due to the direct action of the pharmaceutical ingredient of the composition or may be due to one or more other materials additively or synergistically interacting with the pharmaceutical ingredient. The present invention provides a slow release therapeutic composition comprising apolactoferrin as an active pharmaceutical ingredient, a binder, a filler and a gelling agent that, as formulated in the therapeutic composition delivered intravaginally for the treatment of bacterial vaginosis. By treatment of bacterial vaginosis, it is meant that the therapeutic composition is effective to prevent or reduce the incidence, severity and/or duration of the infection. The therapeutic composition may also prevent reoccurrence of the infection.

Apolactoferrin is a protein derived and processed from lactoferrin to decrease its iron content from approximately 15% saturation of its iron binding sites in native cows' milk, to less than 4% saturation levels in the Apo form. Bacteria have evolved highly specialized lactoferrin binding receptors, that are specific to human lactoferrin and can extract iron directly from human lactoferrin. These same receptors will not bind bovine lactoferrin since there is only 70% homology of the protein sequence as compared with the human form. It is proposed that using apolactoferrin with lower iron will result in less available iron to bacteria since with sufficient excess, it will draw down the level of human lactoferrin iron to essentially 1-4% as compared to saturations of 15-30 percent of human lactoferrin that may exist in the vaginal environment. Reduction of bound iron to human lactoferrin to such low levels can never be accomplished by addition of even a large molar excess of 15% bovine lactoferrin, since the human levels of lactoferrin saturation will never go lower than the bovine lactoferrin that is competing with it. This understanding of the specific needs of bacterial vaginosis makes apolactoferrin an interesting form of bovine lactoferrin for treatment of bacterial vaginosis and has not previously been described. Not to be bound by theory but to aid in the understanding of the invention, using apolactoferrin with low iron saturation will result in an order or magnitude less available iron to bacteria which cause BV as compared to native bovine lactoferrin with an approximately 15% iron level.

In another embodiment the therapeutic composition containing apolactoferrin contains a second active pharmaceutical ingredient for example proteins, peptides, minerals, antibiotics, anti-inflammatories, cytokines, hormones, anesthetics, analgesics and fragments thereof. In a further embodiment, the therapeutic composition containing apolactoferrin can be administered concomitantly with another pharmaceutical ingredient for example proteins, peptides, minerals, antibiotics, anti-inflammatories, cytokines hormones, anesthetics, analgesics and fragments thereof.

In another embodiment, the therapeutic composition containing apolactoferrin can be administered before administration of another pharmaceutical ingredient for example proteins, peptides, minerals, antibiotics, anti-inflammatories, cytokines hormones, anesthetics, analgesics and fragments thereof.

In a further embodiment, the therapeutic composition containing apolactoferrin can be administered following administration of another pharmaceutical ingredient for example proteins, peptides, minerals, antibiotics, anti-inflammatories, cytokines, hormones, anesthetics, analgesics and fragments thereof.

In a further embodiment, the therapeutic composition containing apolactoferrin can be administered before, concomitantly or following administration of an antibiotic. The use of antibiotics is considered the standard of care treatment for bacterial vaginosis. Non limiting examples of antibiotics used include amoxicillin, ampicillin, ceftiofur, metronidazole, secnidazole, tinidazole, lincosamides, and clindamycin. Treatment with apolactoferrin, which has a different mechanism of action than traditional antibiotics may work additively or synergistically with these antibiotics and additionally may eliminate residual bacteria following antibiotic treatment via iron deprivation and biofilm disruption. The therapeutic composition as envisioned could be administered in support of antibiotic therapy, being synergetic and possibly improve efficacy of the antibiotic treatment.

Besides bacteria binding and extracting iron from lactoferrin, there is an additional, well described mechanism bacteria use to obtain iron. That mechanism is siderophores. These relatively low molecular weight (approximately 500-1,000 Dalton) specialized molecules are manufactured and secreted by many bacterial species. Hundreds of different types of siderophores exist. They are classified in several groups based on structural similarities. They all bind iron with a great affinity, often with greater affinity for iron than lactoferrin. This permits them to steal iron from lactoferrin and present this iron to the bacteria. However, considering the higher metabolic overhead to bacteria to manufacture and secrete siderophores, some of which are never retrieved by the bacteria, siderophores function only as a second tier for iron acquisition that is used only when iron cannot be obtained directly from human lactoferrin via the specially evolved, species-specific lactoferrin binding proteins. When iron acquisition via siderophores is needed, siderophores are manufactured, secreted and bind iron outside of the bacterium, they are then taken up and internalized into the bacterium via specialized periplasmic binding proteins. The manufacture of siderophores by Gardnerella vaginalis, the most prevalent pathogen in bacterial vaginosis, is well described. To counter bacterial siderophores, and as part of the cat-and-mouse struggles for iron, the body manufactures “anti-siderophore” compounds that bind siderophores, even those loaded with iron and thus prevent uptake by bacteria. The protein described is lipocalin2 (LCN2), a 25 kDa compound that has specific high affinity for the “catecholate-type” siderophores that Gardnerella produces including vibriobactin, enterobactin and bacillibactin. This protein is well described and is alternatively named Neutrophil Gelatinase-Associated Lipocalin (NGAL), or siderocalin. Lipocalin 2 (LCN2) is an antimicrobial protein present in milk and other biological fluids that withholds iron from bacterial pathogens by binding their iron-scavenging siderophores. In theory, these siderophores may provide an escape mechanism for Gardnerella vaginalis and other BV-associated bacteria to acquire iron even in the presence of bovine lactoferrin. By adding this mechanism of iron sequestration, we hypothesize that LCN2 may enhance the antimicrobial effects of bLF by providing a second mechanism for depriving bacteria of iron and act alone or synergistically with bLF. We have isolated, purified, and tested Bovine LCN2 and found it to be functional against the common siderophores.

In an embodiment the pharmaceutical ingredient is lipocalin-2. In an embodiment the therapeutic composition comprises lipocalin-2, as the active pharmaceutical ingredient, a binder and a filler that, as formulated in the therapeutic composition, presents a therapeutic effect in human hosts. The therapeutic composition delivered intravaginally, containing lipocalin-2 in a therapeutic composition can be formulated as immediate release or a slow release composition. The present invention provides a therapeutic composition comprising liocalin-2, as the active pharmaceutical ingredient, a binder, a filler and a gelling agent delivered intravaginally for the treatment of bacterial vaginosis. In that regard, the therapeutic effect may be due to the direct action of the pharmaceutical ingredient of the composition or may be due to one or more other materials synergistically interacting with the pharmaceutical ingredient. The present invention provides a therapeutic composition delivered intravaginally for the treatment of bacterial vaginosis. By treatment of bacterial vaginosis, it is meant that the therapeutic composition is effective to prevent or reduce the incidence, severity and/or duration of the infection. The therapeutic composition may also prevent reoccurrence of infection.

In another embodiment the therapeutic composition containing lipocalin-2 can be administered with a second active pharmaceutical ingredient for example proteins, peptides, minerals, antibiotics, anti-inflammatories, cytokines hormones, anesthetics, analgesics and fragments thereof. In an embodiment the second active pharmaceutical ingredient is apolactoferrin.

In a further embodiment, the therapeutic composition containing lipocalin-2 can be administered concomitantly with another pharmaceutical ingredient for example proteins, peptides, minerals, antibiotics, anti-inflammatories, cytokines, hormones, anesthetics, analgesics and fragments thereof.

In another embodiment, the therapeutic composition containing lipocalin-2 can be administered before administration of another pharmaceutical ingredient for example proteins, peptides, minerals, antibiotics, anti-inflammatories, cytokines, hormones, anesthetics, analgesics and fragments thereof.

In a further embodiment, the therapeutic composition containing lipocalin-2 can be administered following administration of another pharmaceutical ingredient for example proteins, peptides, minerals, antibiotics, anti-inflammatories, cytokines, hormones, anesthetics, analgesics and fragments thereof.

In a further embodiment, the therapeutic composition containing lipocalin-2 can be administered before, concomitantly or following administration of an antibiotic. In an embodiment the therapeutic composition containing lipocalin-2 is administered following antibiotic treatment for bacterial vaginosis.

In an embodiment the therapeutic composition comprises lactoferrin, lipocalin-2, a bio-adhesive polymer, binder, lubricant, glidant and filler that, as formulated in the therapeutic composition, presents a therapeutic effect in human hosts, for the treatment of bacterial vaginosis either alone or in combination with other materials. In that regard, the therapeutic effect may be due to the direct action of the pharmaceutical ingredient of the composition or may be due to one or more other materials additively or synergistically interacting with the pharmaceutical ingredient. The present invention provides a therapeutic composition delivered intravaginally for the treatment of bacterial vaginosis. By treatment of bacterial vaginosis, it is meant that the therapeutic composition is effective to prevent or reduce the incidence, severity and/or duration of the infection. The therapeutic composition may also prevent reoccurrence of the infection.

Not to be bound by theory, but to aid in the understanding of the invention, the combination of lipocalin-2 and lactoferrin may increase the antimicrobial effects of lactoferrin by providing a second mechanism for depriving bacteria of iron. In cases where bacteria secrete siderophores as an alternative strategy to access iron, we have included lipocalin 2, a siderophore-neutralizing protein in our compositions.

The therapeutic composition of the present invention may be administered to a patient to achieve any desired effect in clinical outcome for the targeted vaginosis. In an embodiment a method is provided for the delivery of therapeutic compositions and dosage forms for treating or preventing diseases or disorders of the vagina. In an embodiment a method is provided for the delivery of slow release therapeutic compositions and dosage forms for the treatment of bacterial vaginosis.

In another embodiment the therapeutic compositions of the present invention may be used for treatment of preterm birth. Vaginal dysbiosis caused by BV is associated with a risk of late miscarriage or preterm birth. Inflammation is another risk factor for preterm birth. Therapeutic compositions that deliver slow release levels of bovine lactoferrin, a protein that has both iron-binding and anti-inflammatory activities, may prevent preterm birth. The therapeutic composition comprises at least one active pharmaceutical ingredient, and at least one excipient that, as formulated in the therapeutic composition, presents a therapeutic effect in human hosts, for the treatment of vaginal dysbiosis either alone or in combination with other materials. The therapeutic composition of the present invention comprises lactoferrin, a bio-adhesive polymer, and at least one excipient that, as formulated in the therapeutic composition, presents a therapeutic effect in human hosts, for the treatment of vaginal dysbiosis either alone or in combination with other materials resulting in the prevention or reduction in occurrence of pre term birth. In that regard, the therapeutic effect may be due to the direct action of the pharmaceutical ingredient of the composition or may be due to one or more other materials synergistically interacting with the pharmaceutical ingredient. The present invention provides a therapeutic composition delivered intravaginally for the treatment of bacterial vaginosis reducing the vaginal dysbiosis associated with preterm birth.

In another embodiment, a kit may comprise the therapeutic compositions described above. In a specific embodiment, the kit may comprise daily active dosage units comprising a pharmaceutical ingredient in a therapeutic composition, herein the therapeutic composition allows for daily dosing regimen as prescribed, and wherein the initial administration of the therapeutic composition establishes its effect on bacterial vaginosis.

In another embodiment a kit may comprise the therapeutic composition of the present invention as well as an antibiotic formulation that is standard of care, thus having the ability to provide both antibiotic and therapeutic composition for daily dosage regimen.

In another embodiment, the present disclosure provides a method for making the slow release composition comprising: combining one or more active pharmaceutical ingredients, a binder and a filler, mixing the ingredients to combine, and pressing the ingredients into a tablet. Excipients can be added to improve the desired characteristics of the tablet.

### EXAMPLES

### Example 1: Formulation Development

Vaginal tablets containing bovine lactoferrin (bLF) for use in manufacturing process development and clinical trials were prepared as follows. All ingredients were sifted through a Sieve #50 then blended via either manual mixing while sieving (250 gram scale) or using a V-blender for 30 minutes (1,000-2,000 gram scale). Tablets were compressed using tablet-presses standard to the industry with gravity feed of the blend. Deep concave bullet shaped tooling for upper and lower punches was used. Tablets were pressed to a hardness of ˜8-12 kP breaking force for use in in vitro experiments and ˜5-12 kP breaking force for use in clinical trials. Representative compositions are shown in Table 1.

To arrive at an optimal tablet, approximately 100 formulations were made in 26 series to arrive at the type of excipients and their precise ratios to accomplish slow dissolution within a low fluid environment. A specialized chamber was constructed to simulate the vaginal environment, both in its total fluid content, surface area and continuous fluid ingress and egress. Prior to human testing in clinical trials, six final formulations labeled Z1-Z6 were evaluated for dissolution testing using the standard USP dissolution test in 900 mL of acetate pH 4.6. In that system and under those conditions the tablets dissolved to completion in 30-60 minutes. However, in a low volume dissolution apparatus (1-2 mL with shaking) designed by us specifically to mimic the limited fluid environment we are exploiting for slow release vaginal drug delivery, it was observed that dissolution was prolonged to greater than 4 hours (data not provided). This observation distinguishes the bovine lactoferrin tablets from other vaginal tablets or suppositories that are designed for rapid dissolution. In our formulations, which contain uniformly blended ingredients throughout the tablet, upon wetting, there is a clear formation of an outer gel. Second, when the outer gel forms, the inner portions of the tablet stay dry and hard, even if it is immersed in excess amounts of fluid. Third, only when the gel is successively dispersed do inner portions of the tablet containing the remaining active pharmaceutical ingredient become wet, as it comes into contact with the dissolving solution. This progressed until final tablet dissolution. In contrast, it is clearly observed that in rapidly dissolving tablets, the tablet wets completely throughout, and often fragments into multiple particles or forms a paste-like watery mass. This traditional mechanism, continuously exposes the active pharmaceutical ingredient to fluid and enhances dissolution of the tablet into the surrounding fluid. Indeed, this mechanism is utilized by the currently available lactoferrin vaginal product (Difesan),

Our formulations Z1-Z6 all dissolved more slowly than a comparator tablet containing a disintegrant (crospovidone). Formulations Z1-Z3, which contain mannitol as a filler, dissolved more quickly that formulations Z4-Z6. Partially wetted tablets Z2, Z5, and Z6 which contain polyvinylpyrrolidone had a stickier tactile feel in vitro and were expected to have better bioadhesive properties in vivo.

Tablet dissolution and rate of release of active pharmaceutical ingredients (APIs) can be further modified by making bilayer tablets. The two sides of the tablet can have different rates of dissolution and can deliver APIs at different rates. For example, the dissolution rate of formulation Z2 and release of lactoferrin from this composition can be prolonged by covering one surface of the tablet with a different filler such as lactose. Formulation Z2X, a bilayer tablet containing 300 mg of lactoferrin is predicted to release lactoferrin more slowly than formulation Z2 because the surface area of lactoferrin accessible to water is decreased by the B side of formulation Z2X. The B side of formulation Z2X contains lactose and manganese and polyvinyl-pyrrolidone as a gelling agent. Thus the B side of formulation Z2X will additionally deliver lactose and manganese to the vaginal environment. The rate of release of API such as manganese from the B side of a bilayer tablet can also be modulated. For example, formulation Z2Y, which contains more gelling agent on the B side than formulation Z2X would be expected to deliver manganese and lactoferrin more slowly.

Bilayer tablet formulation Z11 was manufactured using blending techniques standard in the art and tableting with using a Riva Piccola DC bilayer tablet press. Tablets met release specifications for hardness and friability and were acceptable for use.

TABLE 1

| | | | | | | | Polyvinyl- | | | | | |
|---|---|---|---|---|---|---|---|---|---|---|---|---|
| | | | | | | | pyrrolidone | | glyceryl | silicon | | |
| | | apo- | | | | | K90 | | dibehenate | dioxide | | |
| | lacto- | lacto- | | | | | (kollidon | poloxamer | (Compritol | (Aerosil | cros- | Lactose |
| ID | ferrin | ferrin | manganese | zinc | lipocalin-2 | mannitol | 90 ®) | 407 | 888 ®) | 972 ®) | povidone | monohydrate |
| Z1 | 300 | — | — | | — | 200 | — | — | 1.00% | — | — | |
| | mg | | | | | mg | | | | | | |
| Z2 | 300 | — | — | | — | 200 | 2.25% | — | 1.00% | — | — | |
| | mg | | | | | mg | | | | | | |
| Z3 | 300 | — | — | | — | 200 | — | 2.25% | 1.00% | — | — | |
| | mg | | | | | mg | | | | | | |
| Z4 | 300 | — | — | | — | — | — | — | 1.00% | — | — | |
| | mg | | | | | | | | | | | |
| Z5 | 300 | — | — | | — | — | 3.60% | — | 1.00% | — | — | |
| | mg | | | | | | | | | | | |
| Z6 | 300 | — | — | | — | — | 3.50% | 3.50% | 1.00% | — | — | |
| | mg | | | | | | | | | | | |
| Z7 | 300 | — | 50 | | — | 200 | 2.25% | — | 2.25% | 0.25% | — | |
| | mg | | μg | | | mg | | | | | | |
| Z8 | 300 | — | — | | — | 200 | 4.00% | — | 2.25% | 0.25% | — | |
| | mg | | | | | mg | | | | | | |
| Z9 | — | 300 | — | | — | 200 | 4.00% | — | 2.25% | 0.25% | — | |
| | | mg | | | | mg | | | | | | |
| Z10 | — | 300 | 50 | | — | 200 | 4.00% | — | 2.25% | 0.25% | — | |
| | | mg | μg | | | mg | | | | | | |
| Z11 | — | — | 50 | | — | 450 | 4.00% | — | 2.25% | 0.25% | — | |
| old | | | μg | | | mg | | | | | | |
| Z12 | — | — | 50 | | — | 450 | 4.00% | — | 2.25% | 0.25% | 1.00% | |
| | | | μg | | | mg | | | | | | |
| Z13 | — | — | — | | 3 | 450 | 4.00% | — | 2.25% | 0.25% | — | |
| | | | | | mg | mg | | | | | | |
| Z14 | — | — | — | | 3 | 450 | 4.00% | — | 2.25% | 0.25% | 1.00% | |
| | | | | | mg | mg | | | | | | |
| Z2 X | 300 | — | — | | — | 200 | 2.25% | — | 1.00% | — | — | — |
| Side A | mg | | | | | mg | | | | | | |
| Z2 X | — | — | 50 | — | — | — | 2.25% | — | 1.00% | — | — | 200 |
| Side B | | | μg | | | | | | | | | mg |
| Z2 Y | 300 | — | — | | — | 200 | 2.25% | — | 1.00% | — | — | — |
| side A | mg | | | | | mg | | | | | | |
| Z2 Y | — | — | 50 | — | — | — | 5.00% | — | 1.00% | — | — | 200 |
| Side B | | | μg | | | | | | | | | mg |
| Z11 | — | 300 | 50 | 250 | — | 200 | 2.25% | — | 1.00% | 0.25% | — | |
| side A | | mg | μg | μg | | mg | | | | | | |
| Z11 | — | — | — | — | — | — | 5.00% | — | 1.00% | — | — | 200 |
| Side B | | | | | | | | | | | | mg |

### Example 2: Pharmacokinetic Studies

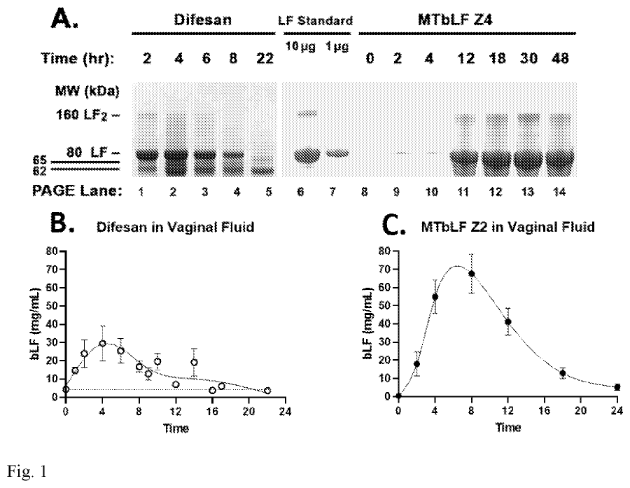

Slow release bovine lactoferrin (bLF) tablets were prepared using the method outlined in Example 1. The vaginal pharmacokinetics (concentration of lactoferrin in vaginal fluid) of bLF formulations or Difesan (a lactoferrin formulation commercially available in Italy) have been evaluated in several separate clinical studies. All studies enrolled healthy women or women with asymptomatic bacterial vaginosis. All tablets contained 300 mg of bovine lactoferrin and were inserted vaginally using an applicator per the manufacturers' instructions. Vaginal swabs were collected pre-dose and over 48 hours post dosing. Vaginal fluid was extracted from the swabs and analyzed for lactoferrin content using C3 reverse phase chromatography, spectrophotometry, or SDS-PAGE analysis as indicated. FIG. 1, Panel A contrasts a representative tablet formulation Z4 (labeled MTbLF Z4) as compared to with Difesan, a marketed lactoferrin formulation that includes a disintegrant. Lanes 1-5 are from a human trial with Difesan, Lanes 9-14 are from a human trial with invention formulation Z4, lanes 6& 7 are lactoferrin standards. The 80 kDa lactoferrin band is indicated at the left. Dimeric (160 kDa) and trimeric (240 kDa) lactoferrin as well as 65 and 62 kDa proteolytic fragments of lactoferrin are also indicated. Difesan levels in vaginal fluid peaked at 4 hour post dose and were waning by 8 hours post dose (FIG. 1, Panel B). In contrast, bLF levels were low but measurable at 2-4 hours, and remained at high sustained levels over 12-48 hour, the last timepoint evaluated. These data demonstrate that formulations without disintegrant dissolve more slowly and that lactoferrin itself can act as a slow-release gelling agent. In order to support once daily administration, lactoferrin can be dispersed within the tablet using a filler such as mannitol in combination with a bioadhesive agent such as polyvinylpyrrolidone K90 to shift the concentration time profile from ˜12-48 hr to ˜2-24 hours. FIG. 1 Panel C shows data with formulation Z2 (labeled MTbLF Z2) where lactoferrin concentrations peaked at approximately 8 hours after administration then waned gradually out to 24 hours post dosing. At 18 hr post dose, bLF was measurable in 15 of 18 samples, mean 12.9±2.5 (mean±SEM) median 11.2 mg/mL while with Difesan only 1 of 10 samples had measurable levels of lactoferrin, 6.1 mg/mL at 17 hr. The bLF Z2 formulation, which does not contain a disintegrant, provided increased exposure of lactoferrin relative to Difesan, a state of the art formulation (compare FIG. 1, Panels B and C). As the data shows, besides extending the therapeutic level for approximately 24 hours, formulation Z2, as compared to Difesan, achieves a greater area under the curve. We believe that this is secondary to complete, yet slow, dissolution of Z2 tablet. Vaginal fluid is constantly turning over during the course of a day, carrying with it some amount of vaginally administered drug whether it is in a gel form, liquid form or undissolved tablet fragments. The slow release tablet formulations should decrease the loss of undissolved active pharmaceutical ingredient via vaginal discharge, compared with tablets that fragment more rapidly and may explain the greater area under the curves with slow release formulations. We embarked on the development of our slow release technology since we found that only by slowing down dissolution can sustained levels of therapeutic be accomplished. Otherwise, therapeutic released by the tablet is invariably eliminated by the vagina with 4-6 hours, almost independent of concentration or mucoadhesiveness.

### Example 3: Synergy with Antibiotics

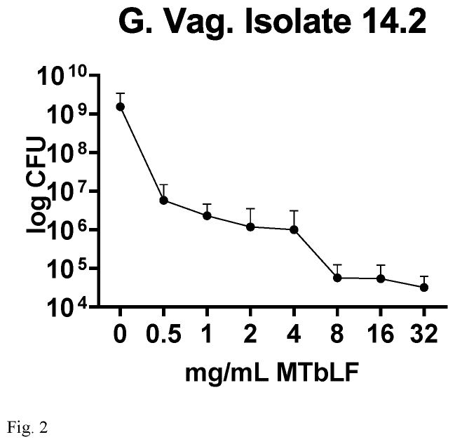

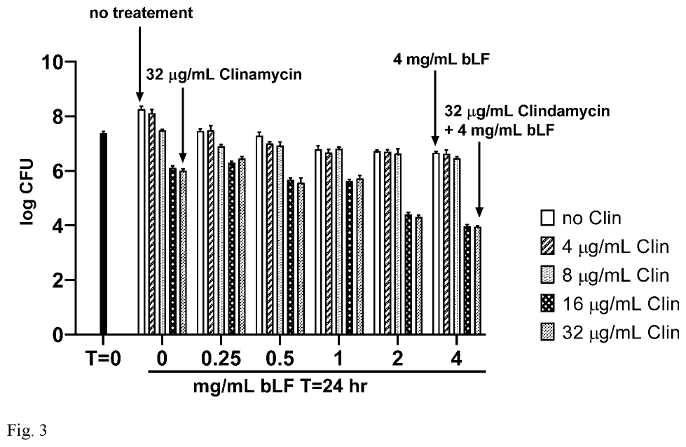

Gardnerella vaginalis (G. vag) is a predominant species bacterial vaginosis (BV). The antimicrobial activity of bovine lactoferrin (bLF) and its ability to synergize with antibiotics was demonstrated using clinical isolates of G. vag and defined medium. G. vag isolates were recovered from the vaginal discharge of BV positive patients and identification at the species level was performed by species-specific PCR using the primer pairs G. vaginalis V2-R1 and G. vaginalis V6-U2. G. vag isolate 14.2 was used for the experiments presented in FIG. 2 and FIG. 3.

Cells were grown in aerobic conditions at 37° C. at 5% CO2 in PMD medium. PMD medium contains: proteose peptone No. 3 (BD211693) 15 g/L, maltone 2 g/L, dextrose 1 g/L, Na2HPO2 1 g/L, NaH2PO4 1 g/L adjusted to pH6.8. High Iron PMD medium additional contains 500 mg/L human hemoglobin. No Iron PMD is prepare by treating PMD medium with Chelex® 100 Chelating Resin (Bio-Rad Cat #1432832) then adding back 0.1 mM magnesium sulfate, 0.1 mM calcium chloride, and 10 μM zinc chloride. Low Iron PMD medium is prepared by adding 10 μM ferric ammonium citrate (FAC) to No Iron PMD. Overnight broth cultures of G. vaginalis were grown in High Iron PMD, centrifuged, resuspended No Iron PMD, incubated for 2 hr at 37° C. then diluted to 2.5×10⁷ cells/mL in Low Iron PMD. Cells were grown in liquid culture for 24-48 hr then plated on agar to assess viability (expressed as colony forming units per mL (CFU)). Bulk bLF was dissolved in water and diluted in culture medium. Formulated tablets were not tested in this in vitro system.

Dose-dependent inhibition of G. vag growth was demonstrated over a concentration range of 0.5-32 mg/mL (FIG. 2). These in vitro data demonstrate that bLF kills BV-associated bacteria over a concentration range consistent with that achieved in vivo following vaginal administration of slow release formulations.

Current standard-of-care treatments for BV rely on antibiotics and all show unacceptably low cure rates and high relapse rates. Moreover, bacterial resistance to antibiotics is a significant problem in BV. We have performed microbiological studies where lactoferrin has not only been effective at treating antibiotic resistant G. vag strains, but also acts in synergy with antibiotics to decrease the dose necessary for bacterial killing. This fact is important in developing antibiotic sparing treatments for BV. Antibiotic synergy testing for G. vag strain 14.2 was performed using a checkerboard cross dilution method using broth cultures according to US National Committee for Clinical Laboratory Standards (NCCLS) publications. Clindamycin and lactoferrin were each tested alone or in combination range from ¼× minimum inhibitory concentration (MIC) up to 2× of the MIC. Results are shown in FIG. 3. Initial levels of G. vag 14.2 were 7.8 CFU/mL and increased to 8.3 CFU/mL after 24 hr. In the presence of 32 mg/mL clindamycin, growth was inhibited 27.4% (6.0 CFU/mL) at 24 hr. In the presence of 4 mg/mL bLF, growth was inhibited 19.3% (6.7 CFU/mL) at 24 hr. The combination of 32 mg/mL clindamycin and 4 mg/mL bLF inhibited growth 52.2% (3.9 CFU/mL). These results indicate that bLF can enhance the antimicrobial effect of antibiotics.

### Example 4: Manganese Supplementation

The human vaginal microbiome is dominated by lactobacilli, which create an acidic environment thought to protect women against sexually transmitted pathogens and opportunistic infections. Bacterial vaginosis is a dysbiotic condition characterized by a shift from normal vaginal microbes—predominantly lactobacilli in a low pH environment to anerobic species like Gardnerella vaginalis and Atopobium vaginae in an elevated pH environment. Our invention uses lactoferrin alone, manganese alone or lactoferrin and manganese in combination to create an environment where lactobacilli flourish and BV-associated bacteria cannot grow. Virtually all bacteria need iron, Lactobacilli are an exception and are not suppressed because they do not require iron, they are able to use manganese instead. In this example we demonstrate that manganese is limiting in BV thus providing the rationale for manganese supplementation as a treatment for BV.

Concentrations of 12-50 μg/mL elemental manganese are commonly used to culture lactobacilli species with 16 μg/mL (ATCC Medium: 416 Lactobacilli MRS Agar/Broth) used most commonly to grow strains such as Lactobacillus crispatus, a protective species in the vaginal microbiome. We used inductively coupled mass spectrometry (ICP-MS) to measure elemental Mn in vaginal fluid from three women whose BV status was unknown. All had manganese levels significantly below that described in the literature as optimal for lactobacilli. Specifically, manganese levels were 0.047 μg/mL, <0.017 μg/mL and 0.039 μg/mL for donors T2407, T5659, and J4265.

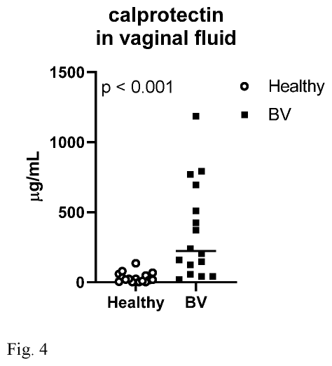

We have found that levels of manganese in vaginal fluid are low and are suppressed even further by the host immune response to BV-associated bacteria. Calprotectin, a protein highly expressed in neutrophils, contributes to this host defense by withholding Mn and Zn from invading pathogens. We compared levels of calprotectin in healthy women (Nugent Score 0-3) and women with BV (Nugent score 8-10). Vaginal swabs were collected, eluted with 1 mL of PBS, and residual solids were removed by centrifugation. Levels of calprotectin were measured by ELISA (QUANTA Lite® Calprotectin ELISA, Inova Diagnostics). As show in FIG. 4, calprotectin levels were 10-fold higher in women with BV than healthy women (median 223 μg/mL vs 22 μg/mL, p<0.001).

Taken together, the observation of low concentrations of manganese in vaginal fluid coupled with elevated calprotectin levels in bacterial vaginosis show a non-ideal environment for growth of lactobacillus and maintenance of a healthy vaginal microbiome and yield proof of concept for use of manganese for treatment of BV.

### Example 5: Manganese and Zinc Supplementation for BV and Maintenance of a Healthy Vaginal Microbiome

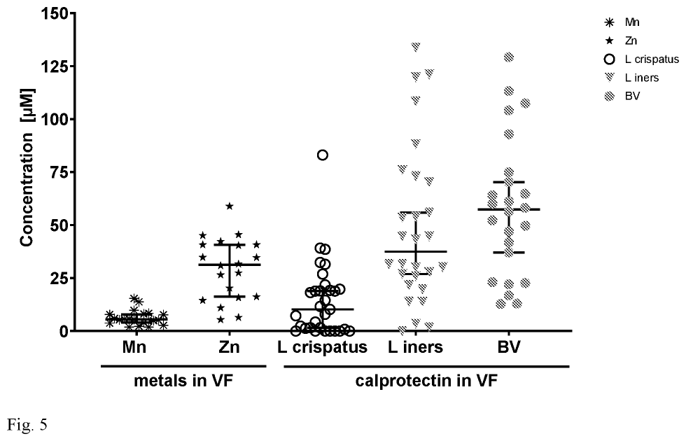

FIG. 5 confirms the observation that vaginal calprotectin levels are increased in women with BV and also demonstrates that calprotectin is elevated in women with L-iners dominated vaginal microbiomes relative to women with healthy microbiomes dominated by L. crispatus. Sequential vaginal swabs were collected for PCR analysis of the vaginal microbiome (AusDiagnostics Vaginitis and Vaginosis 12-well test (REF87124) and 16S rRNA gene sequencing) and determination of calprotectin levels by ELISA (QUANTA Lite® Calprotectin ELISA, Inova Diagnostics). Vaginal fluid used for ELISA was isolated by centrifugation then diluted. Median concentrations of calprotectin in vaginal fluid were 57 μM in BV (n=24), 37 μM in L. iners (n=28) and 10 μM in BV (n=31) with 95% confidence interval of calprotectin L. iners dominated microbiomes overlapping with 95% confidence interval of calprotecin concentrations in BV. One molecule of calprotectin can bind 2 molecules of zinc or 1 molecule of manganese. When both Mn and Zn are present, Zn is preferentially bound. Levels of manganese and zinc were measured in vaginal fluid from 20 women with unknown BV status. Vaginal swabs were centrifuged in Costar® Spin-X® Centrifuge Tube Filters, 0.22 μm Pore CA Membrane and the cell free fraction of vaginal fluid was analyzed by ICP-MS. The elevated levels of calprotectin in BV and L. iners-dominated vaginal microbiomes were more than sufficient to bind all the available Mn and Zn in vaginal fluid. These results demonstrate that both BV and L. iners create vaginal environments where L. crispatus, which requires Mn not Fe for growth, cannot thrive. L. iners is viewed as a transitional bacteria and women with L. iners-dominated vaginal microbiomes are more likely to have a BV recurrence than women with L. crispatus-dominated vaginal microbiomes. These results yield proof for the the concept of manganese alone or in conjunction with zinc, for the treatment of BV. Furthermore, these results yield proof for the concept of manganese alone or in conjunction with zinc, for the prevention of recurrence of BV and maintenance of a healthy vaginal microbiome.

### Example 6: Doses of Manganese and Zinc for Treatment of BV and Maintenance of a Healthy Vaginal Microbiome

Lactobacillus crispatus has an absolute nutritional requirement for manganese. Elevated levels of calprotectin in BV and in L. iners-dominated vaginal microbiomes sequester manganese and zinc, preventing growth of L. crispatus its maintenance of a healthy vaginal microbiome. Dose ranges for Mn and Zn supplementation were determined by modeling the amount of Mn and Zn delivered from slow release formulations over time and superimposing that model on actual measured concentrations of endogenous levels of Mn and Zn as well as amounts of Mn and Zn that calprotectin will sequester (data from Example 5 and FIG. 5). The pharmacokinetic profile of formulation Z2, which is shown for lactoferrin in FIG. 1C, was used to build the model for Mn and Zn levels in vaginal fluid. Six women each received 3 doses of formulation Z2 separated by 48 hours. Vaginal fluid swabs were collected at t=0, 2, 4, 8, 12, 18, 24, 30, 36, and 42 hours post dose and vaginal fluid concentrations of lactoferrin and mannitol were determined. Average concentrations for the combined 18 dose administrations were obtained based on calculation of Area Under Curve for the locally estimated scatterplot smoothing (Loess) profile of lactoferrin and mannitol concentrations assuming 6 mL volume of distribution. The average concentration of lactoferrin was 33 mg/mL and the average concentrations of mannitol was 20 mg/mL. Average concentrations of both substances correspond to 0.1 fraction or 10% of the administered dose, 300 mg lactoferrin and 195 mg mannitol per Z2 tablet respectively. The 0.1 factor was used to estimate the expected average elemental Mn concentration based on the initial dose per tablet.

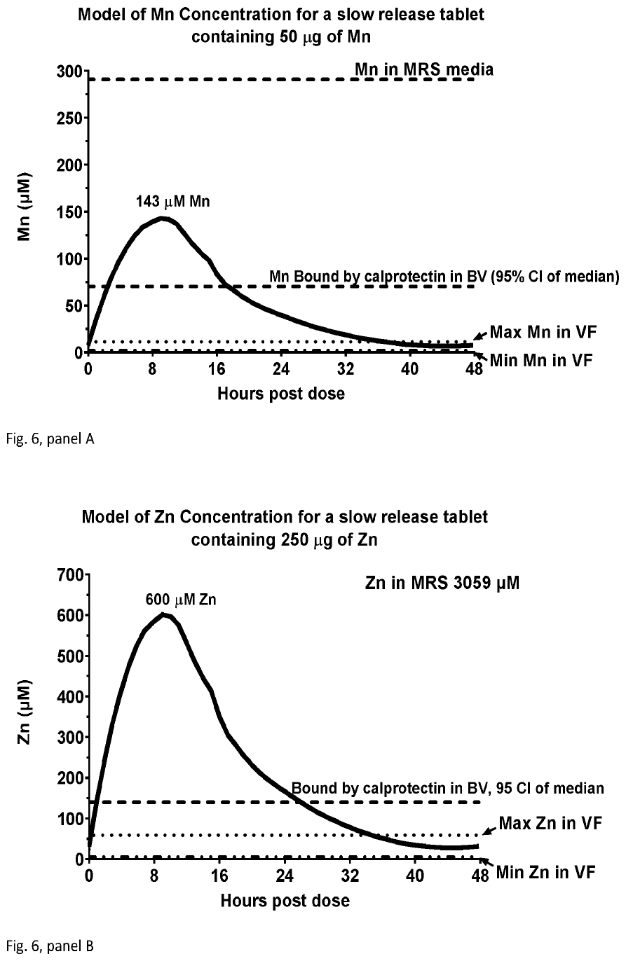

FIG. 6 panel A shows the results of modeling a 50 μg dose of Mn included in a Z2 formulation. A 50 μg dose of Mn in yields a predicted maximal concentration (Cmax) of 143 μM Mn in vaginal fluid, which is ˜2 fold above the amount of Mn that could be bound by calprotectin in BV (95% CI of median is 70 μM based on 1:1 Mn/CP) yet ˜2 fold below the concentration of Mn used in MRS media selective for lactobacillus. A 50 μg dose of Mn also fails to maintain for 24 hours levels Mn above that which can be sequestered by calprotectin (assuming no zinc available to displace Mn from calprotectin). These results indicate doses between the range of 25 and 500 μg of Mn in a slow release formulation are suitable for maintaining and healthy, L. crispatus-dominated vaginal microbiome. It is also possible that even higher levels may provide additional benefit to the vaginal microbiota as other mechanisms of Mn utilization, sequestration or clearance have not been taken into account.

FIG. 6 panel B shows the results of modeling a 250 μg dose of Zn included in a Z2 formulation. A 250 μg dose of Zn in yields a predicted maximal concentration (Cmax) of 600 μM Mn in vaginal fluid, which is ˜4 fold above the amount of Zn that could be bound by calprotectin in BV (95% CI of median is 140 μM based on 2:1 Zn/CP) yet ˜5 fold below the concentration of Zn used in MRS media selective for lactobacillus. A 250 μg dose of Zn also just minimally maintains Zn levels for 24 hours that which can be sequestered by calprotectin. These results indicate doses between the range of 250 and 5000 μg of Zn in a slow release formulation are suitable for maintaining and healthy, L. crispatus-dominated vaginal microbiome. It is also possible that even higher levels of Zn may provide additional benefit to the vaginal microbiota as other mechanisms of Zn utilization, sequestration or clearance have not been taken into account.

### Example Embodiment Combinations

Some other contemplated embodiment combinations for various aspects of this disclosure, with or without additional features as disclosed above or elsewhere herein, are summarized in the numbered paragraphs presented below, and in the appended claims:

1. A product for use to treat for bacterial vaginosis in a human, the product comprising:
at least one administration unit configured for intravaginal administration, wherein a said administration unit comprises in a water-soluble form at least one active pharmaceutical ingredient for treatment of bacterial vaginosis selected from the group consisting of:
   essential metal at a therapeutically effective dose of essential metal for treatment of bacterial vaginosis, wherein the essential metal is selected from the group consisting of manganese at a therapeutically effective dose of manganese (preferably in a range of from 10 to 1000 micrograms of manganese), zinc at a therapeutically effective dose of zinc (preferably in a range of from 25 micrograms to 5000 micrograms of zinc) and combinations thereof
   iron-depleted lactoferrin, at a therapeutically effective dose of iron-depleted lactoferrin for treatment of bacterial vaginosis and preferably in a range of from 50 to 500 milligrams of iron-depleted lactoferrin;
   lipocalin-2, at a therapeutically effective dose of lipocalin-2 for treatment of bacterial vaginosis and preferably in a range of from 0.3 to 30 milligrams of lipocalin-2; and
   combinations thereof. In some preferred implementations, the essential metal comprises manganese, alone or in combination with zinc.

2. A product for use to supplement vaginal bacterial health in a human, optionally to supplement nutritional immunity to bacterial infection associated with bacterial vaginosis, the product comprising:
at least one administration unit configured for intravaginal administration, wherein a said administration unit comprises in a water-soluble form of at least one vaginal health supplement ingredient selected from the group consisting of:
   essential metal at a health supplement dose of essential metal, the essential metal being selected from the group consisting of manganese at a health supplement dose of manganese (preferably in a range of from 10 to 1000 micrograms of manganese), zinc at a health supplement dose of zinc (preferably in a range of from to 5000 micrograms of zinc) and combinations thereof
   iron-depleted lactoferrin, at a health supplement dose of iron-depleted lactoferrin and preferably in a range of from 50 to 500 milligrams of iron-depleted lactoferrin;
   lipocalin-2, at a health supplement dose of lipocalin-2 and preferably in a range of from 0.3 to 30 milligrams of lipocalin-2; and
   combinations thereof. In some preferred implementations, the essential metal comprises manganese, alone or in combination with zinc.

3. The product of either one of paragraph 1 or paragraph 2, wherein the said administration unit comprising the ingredient is in a form of a vaginal suppository tablet.

4. The product of paragraph 3, wherein the vaginal suppository tablet is formulated to dissolve from the vaginal suppository tablet all of each said ingredient included in the vaginal suppository tablet over a time period of from 20 to 90 minutes, and preferably from 30 minutes to 90 minutes, according to dissolution test procedure of U.S. Pharmacopeia <711> into 900 mL of dissolution medium of 0.2M acetate buffered solution at pH 4.5±0.05 and 37±0.5° C. in a basket dissolution apparatus with 1 liter vessel volume and mixing speed of 75 revolutions per minute, with 5 mL syringe samples of dissolution medium collected at intervals of 15, 30, 45, 60, 90 and 120 minutes and analyzed to determine extent of dissolution of target component.

5. The product of any one of either one of paragraph 3 or paragraph 4, wherein the vaginal suppository tablet is formulated to dissolve from the vaginal suppository tablet all of each said ingredient included in the vaginal suppository tablet over a time period of from 2 to 10 hours, and preferably from 3 to 10 hours, under the following conditions:
a 2 milliliter sample of normal saline as a dissolution solution at 37° C. is placed into a 5 milliliter tube;
a tablet is placed in the tube containing the dissolution solution and the tube is sealed and secured to a rotary shaker;
the rotary shaker with the secured tubes is operated at a 30 degree tilt, speed of turns per minute and temperature setting of 37° C.;
the shaker is operated for 10 hours, pausing briefly for intermediate visual observation of tube contents at 0.5, 1, 2, 4 and 8 hours;
dissolution status of the tablet is record for each time point of 0.5, 1, 2, 4 and 10 hours and the time point is recorded at which total tablet dissolution occurs or that total tablet dissolution did not occur during the 10 hour test period as determined by visual observation.

6. The product of any one of paragraphs 3-5, wherein the vaginal suppository tablet comprises a gelling agent dispersed in the vaginal suppository tablet, and preferably the gelling agent is a bioadhesive gelling agent.

7. The product of paragraph 6, wherein the gelling agent is selected from the group consisting of acacia, alginic acid, bentonite, carbomers, carboxymethyl cellulose, ethylcellulose, gelatin, hydroxyethylcellulose, hydroxypropylcellulose, magnesium aluminum silicate, methylcellulose, poloxamers, polyvinyl alcohol, sodium alginate, tragacanth, xanthan gum, poloxamer 407, poloxamer 188, polyvinylpyrrolidone, copovidone (vinylpyrrolidone-vinyl acetate copolymer) (e.g., Kollidon® VA 64, BASF), celluloses, chitosans, mucin glycoproteins, trefoil peptides, polycarbophils and combinations thereof, and preferably with the gelling agent selected from the group consisting of poloxamer 407, poloxamer 188, polyvinylpyrrolidone, copovidone (e.g., Kollidon® VA 64, BASF), celluloses, chitosans, mucin glycoproteins, trefoil peptides, polycarbophils and combinations thereof, and more preferably with the gelling agent selected from the group consisting of celluloses, polyvinylpyrrolidone, copovidone (e.g., Kollidon® VA 64, BASF), and poloxamer 407.

8. The product of either one of paragraph 6 or paragraph 7, wherein the vaginal suppository tablet comprises the gelling agent at a concentration in a range of from 0.5 to 10 weight percent, and with one more preferred range for the concentration of the gelling agent being from 1 to 5 weight percent and another even more preferred range being from 2 to 5 weight percent.

9. The product of any one of paragraphs 3-8, wherein the vaginal suppository tablet is either in the absence of a disintegrant or contains a disintegrant only on one side of a bilayer tablet

10. The product of any one of paragraphs 3-9, wherein the vaginal suppository tablet is essentially in the absence of each of the following: crosslinked polymers, including crosslinked polyvinylpyrrolidone (crospovidone), crosslinked sodium carboxymethyl cellulose (croscarmellose sodium), and the modified starch sodium starch glycolate.

11. The product of paragraph 10, wherein the vaginal suppository tablet comprises from 0.01 to 20 weight percent of a binder dispersed in the vaginal suppository tablet, and with one more preferred range for the concentration of the binder being from 0.05 to 10 weight percent.

12. The product of paragraph 11, wherein the binder is selected from the group consisting of lactose, sucrose, mannitol, microcrystalline cellulose, maltitol, sorbitol, xylitol, water-soluble hydroxyalkyl celluloses, polyvinylpyrrolidone, povidone, xanthan gum, celluloses (e.g., hydroxypropylmethylcellulose, hydroxypropyl cellulose) gelatin, starch, polyethylene glycol and combinations thereof, and preferably with the binder selected from the group consisting of lactose, sucrose, mannitol, microcrystalline cellulose, maltitol, sorbitol, xylitol and combinations thereof, and with one particularly preferred binder being mannitol.

13. The product of any one of paragraphs 3-12, wherein the vaginal suppository tablet comprises up to 80 weight percent filler dispersed in the vaginal suppository tablet, and with one more preferred range for the concentration of the filler being up to 40 weight percent.

14. The product of paragraph 13, wherein the filler comprises filler material selected from the group consisting of lactose, sucrose, mannitol, magnesium stearate, glucose, plant cellulose, calcium carbonate and combinations thereof, and preferably with the filler material being mannitol.

15. The product of any one of paragraphs 3-14, wherein the vaginal suppository tablet comprises from 0 to 50 weight percent sugar or sugar alcohol dispersed in the vaginal suppository tablet, and with one more preferred range for the concentration of the sugar being from 20 to 40 weight percent.

16. The product of paragraph 15, wherein the sugar or sugar alcohol is selected from the group consisting of lactose, sucrose, mannitol, maltitol, sorbitol, xylitol and combinations thereof, and preferably with the filler material selected from the group consisting of lactose, sucrose, mannitol and combinations thereof, and with one particularly preferred filler material being lactose.

17. The product of any one of paragraphs 3-16, wherein the vaginal suppository tablet comprises from 0.25 to 5 weight percent lubricant dispersed in the vaginal suppository tablet, and with one more preferred range for the concentration of the lubricant being from 0.5 to 3 weight percent.

18. The product of paragraph 17, wherein the lubricant is selected from the group consisting of metallic salts of fatty acids (Magnesium stearate, calcium stearate, and zinc Stearate), fatty acids (e.g. stearic acid) fatty acid esters (e.g. glyceride esters (glyceryl monostearate, glyceryl tribehenate, and glyceryl dibehenate) and sugar esters (e.g., sorbitan monostearate and sucrose monopalmitate) and combinations thereof, and preferably with the lubricant selected from the group consisting of fatty acid esters and combinations thereof, and with one particularly preferred lubricant being glyceryl dibehenate.

19. The product of any one of paragraphs 3-18, wherein the vaginal suppository tablet comprises from 0 to 5 weight percent glidant dispersed in the vaginal suppository tablet, and with one more preferred range for the concentration of the glidant being from 0.25 weight percent to 2 weight percent.

20. The product of paragraph 19, wherein the glidant is selected from the group consisting of talc, colloidal silicon dioxide, magnesium stearate, silica and combinations thereof, and with one particularly preferred glidant being silicon dioxide, and preferably a colloidal silicon dioxide, such as for example fumed silica.

21. The product of any one of paragraphs 1-20, comprising a vaginal applicator configured to carry the administration unit, and preferably a vaginal suppository tablet, during insertion into a vagina for intravaginal placement of the administration unit.

22. The product of any one of paragraph 21, wherein the vaginal applicator comprises;
a barrel;
a receptacle disposed at a distal end of the barrel, the receptacle configured to receive a said administration unit, and preferably a said vaginal suppository tablet;
a plunger with a distal end disposed in the barrel and proximal end disposed proximal of the barrel, the plunger being translatable through the barrel to expel the administration unit from the receptacle for intravaginal placement of the administration unit.

23. The product of paragraph 22, wherein the barrel has a longitudinal length of at least 100 millimeters and preferably at least 150 millimeters.

24. The product of paragraph 23, wherein the longitudinal length of the barrel is not larger than 250 millimeters and preferably not longer than 200 millimeters.

25. The product of any one of paragraphs 21-24, comprising a said administration unit, and preferably a said vaginal suppository tablet, received by the vaginal applicator for intravaginal placement.

26. The product of paragraph 25, comprising a plurality of said administration units, and preferably a plurality of said vaginal suppository tablets, each received in a different one of a plurality of said vaginal applicators for intravaginal placement.

27. The product of paragraph 26, wherein each said vaginal applicator having a said administration unit received therein for intravaginal placement is individually enclosed in a separate enclosure, preferably a hermetically sealed enclosure, for example a sealed plastic bag or plastic shrink wrap.

28. The product of any one of paragraphs 21-27, wherein each said vaginal applicator and each said administration unit are contained in a common packaging enclosure.

29. The product of any one of paragraphs 1-28, comprising at least two different said ingredients each selected from the group consisting of:
the essential metal, preferably comprising the dose of manganese;
the iron-depleted lactoferrin; and
the lipocalin-2.

30. The product of any one of paragraphs 1-28, comprising at least two different said ingredients each contained in different said administration units, wherein the at least two different said ingredients are each selected from the group consisting of:
the essential metal, preferably comprising the dose of manganese;
the iron-depleted lactoferrin; and
the lipocalin-2.

31. The product of any one of paragraphs 1-28, comprising at least two different said ingredients contained within a same said administration unit, and more preferably in a same vaginal suppository tablet, wherein the at least two different said ingredients are each selected from the group consisting of:
the essential metal, preferably comprising the dose of manganese;
the iron-depleted lactoferrin; and
the lipocalin-2.

32. The product of paragraph 31, comprising a plurality of the administration units, preferably vaginal suppository tablets, each comprising the at least two different said ingredients, preferably at least 3 and preferably at least 5 of the administration units each comprising the at least two different said ingredients, more preferably from 3 to 100 of the administration units each comprising the at least two different said ingredients, and sometimes from 7 to 10 of the administration units each comprising the at least two different said ingredients. For a week-long treatment, one preferred product includes 7 of the administration units for daily administration over that period. For continued treatment of a chronic condition the number of administration units could exceed 100, for example to provide several months of supply.

33. The product of paragraph 32 wherein the plurality of the administration units each comprising the at least two different ingredients are contained in a common packaging enclosure, and optionally each said administration unit of the plurality of administration units is individually contained in a separate enclosure, preferably a separate hermetically sealed enclosure, for example in a separate sealed cavity of a tray or blister pack comprising the plurality of the administration units.

34. The product of any one of paragraphs 29-33, wherein the at least two different said ingredients comprise the essential metal, preferably comprising the dose of manganese, and the iron-depleted lactoferrin.

35. The product of any one of paragraphs 29-34, wherein the at least two different said ingredients comprise the essential metal, preferably comprising the dose of manganese, and the lipocalin-2.

36. The product of any one of paragraphs 29-35, wherein the at least two different said ingredients comprise the iron-depleted lactoferrin and the lipocalin-2.

37. The product of any one of paragraphs 29-36, comprising the essential metal, preferably comprising the dose of manganese, the iron-depleted lactoferrin and the lipocalin-2.

38. The product of any one of paragraphs 1-37, comprising a said administration unit, preferably a vaginal suppository tablet, comprising the essential metal, preferably comprising the dose of manganese.

39. The product of paragraph 38, wherein the administration unit comprises the dose of manganese in a range having a lower limit selected from the group consisting of 10 micrograms, 20 micrograms, 30 micrograms or 50 micrograms and an upper limit selected from the group consisting of 1000 micrograms, 900 micrograms, 800 micrograms or 600 micrograms. One preferred range for the dose of manganese is from 20 micrograms to 900 micrograms, and one more preferred rage for the dose of manganese is from 30 micrograms to 800 micrograms.

40. The product of either one of paragraph 38 or paragraph 39, comprising a plurality of the administration units, preferably vaginal suppository tablets, each comprising a said dose of essential metal, and preferably a said dose of manganese.

41. The product of paragraph 45, comprising at least 3 and preferably at least 5 of the administration units each comprising a said dose of essential metal, preferably from 5 to 100 of the administration units each comprising a said dose of essential metal, and preferably the dose of manganese, and sometimes from 7 to 10 of the administration units each comprising a said of essential metal, and preferably the dose of manganese. For a week-long treatment, one preferred product includes 7 of the administration units for daily administration over that period. For continued treatment of a chronic condition the number of administration units could exceed 100, for example to provide several months of supply.

42. The product of any one of paragraphs 38-410, wherein the water-soluble form of the essential metal comprises a salt selected from the group consisting of manganese salt, zinc salt and combinations thereof.

43. The product of paragraph 42, wherein;
when the salt comprises a manganese salt, the manganese salt is selected from the group consisting of manganese gluconate, manganese sulfate, manganese chloride, manganese citrate, manganese picolinate and combinations thereof, and with one preferred manganese salt being manganese sulfate; and
when the salt comprises a zinc salt, the zinc salt is preferably zinc sulfate.

44. The product of any one of paragraphs 38-43, wherein the water-soluble form of the essential metal comprises a chelate selected from the group consisting of manganese chelate, zinc chelate and combinations thereof.

45. The product of paragraph 44, wherein when the essential metal comprises the manganese, the manganese chelate is an amino acid chelate, preferably selected from the group consisting of manganese bisglycinate chelate, manganese glycinate chelate, manganese aspartate and combinations thereof.

46. The product of any one of paragraphs 38-45, wherein the essential metal comprises the manganese, and the manganese in water-soluble form is not bound to lactoferrin.

47. The product of any one of paragraphs 38-46, wherein the manganese is in the form of Mn(II).

48. The product of any one of paragraphs 1-47, comprising an iron scavenger contained in an intravaginally administrable form.

49. The product of paragraph 48, wherein the iron scavenger is lactoferrin, preferably bovine lactoferin.

50. The product of either one of paragraph 48 or paragraph 49, wherein the intravaginally administrable form comprises an amount of the iron scavenger with available iron scavenging capacity in a range of from 50 to 500 micrograms of iron. This may represent open iron binding capacity to compete for iron in the vaginal environment, even if not all binding capacity is used following intravaginal administration. This level of available iron binding capacity may provide a large excess relative to available iron in vaginal fluid, and provide a large capacity to compete with iron acquisition mechanisms of undesirable bacteria associated with BV for available iron.

51. The product of any one of paragraphs 48-50, wherein the intravaginally administrable form is an intravaginal suppository tablet comprising the iron scavenger.

52. The product of paragraph 51, wherein the intravaginal suppository tablet is a said administration unit comprising the iron scavenger also comprising the essential metal, preferably the manganese.

53. The product of either one of paragraph 51 or paragraph 52, wherein the intravaginal suppository tablet comprising the iron scavenger is a said administration unit also comprising the lipocalin-2.

54. The product of any one of paragraphs 1-53, comprising a dose of lactoferrin contained in an intravaginally administrable form.

55. The product of paragraph 54, wherein the dose of lactoferrin is in a range of from 50 milligrams to 500 milligrams, and preferably in a range of from 100 to 500 milligrams and more preferably in a range of from 200 milligrams to 400 milligrams.

56. The product of either one of paragraph 54 or paragraph 55, wherein the lactoferrin is selected from the group consisting of human lactoferrin, bovine lactoferrin, recombinant lactoferrin and combinations thereof.

57. The product of any one of paragraphs 54-56, wherein the lactoferrin is bovine lactoferrin.

58. The product of any one of paragraphs 54-57, wherein the lactoferrin is the iron-depleted lactoferrin and the intravaginally administrable form is a said administration unit, preferably a vaginal suppository tablet, comprising the dose of iron-depleted lactoferrin. Intravaginal administration of lactoferrin having 1% or less iron saturation in a sustained release formulation may be expected to provide a concentration in vaginal fluid several times, or even an order of magnitude larger, than a native level of human lactoferrin that may often be present even at elevated levels in people with bacterial vaginosis, with may still be only on the order of perhaps 40 micrograms per milliliter. Accordingly, iron saturation in native lactoferrin should equilibrate at near the iron saturation level of the administered lactoferrin, which may for example be around 1% when administering apo lactoferrin with 1% iron saturation. If the administered lactoferrin is bovine lactoferrin, the iron transferred from the native lactoferrin may become much less available for use by undesirable bacteria that are not able to directly access iron bound to bovine lactoferrin.

59. The product of any one of paragraphs 1-58, wherein a said administration unit, preferably a vaginal suppository tablet, comprises the iron-depleted lactoferrin.

60. The product of paragraph 59, wherein the iron-depleted lactoferrin comprises a level of saturation with bound iron, in increasing order of preference, of not larger than 15 percent, not larger than 10 percent, not larger than 5 percent, not larger than 4 percent, not larger than 3 percent, not larger than 2 percent and most preferably not larger than 1 percent.

61. The product of paragraph 60, wherein the iron-depleted lactoferrin comprises a level of saturation with bound iron of at least 0.2 percent, and often at least 0.5 percent.

62. The product of any one of paragraphs 58-61, wherein the iron-depleted lactoferrin comprises a level of saturation with bound manganese of not more than 1 percent, preferably not larger than 0.5 percent and more preferably the iron-depleted lactoferrin is essentially in the absence of manganese bound to the iron-depleted lactoferrin.

63. The product of any one of paragraphs 58-62, wherein the iron-depleted lactoferrin is selected from the group consisting of human lactoferrin, bovine lactoferrin, recombinant lactoferrin and combinations thereof.

64. The product of any one of paragraphs 58-63, wherein the iron-depleted lactoferrin is bovine lactoferrin.

65. The product of any one of paragraphs 58-64, comprising a plurality of the administration units, preferably vaginal suppository tablets, each comprising a said dose of iron-depleted lactoferrin.

66. The product of paragraph 65 comprising at least 3 and preferably at least 5 of the administration units each comprising a said dose of iron-depleted lactoferrin, preferably from 3 to 100 of the administration units each comprising a said dose of iron-depleted lactoferrin, and sometimes from 7 to 10 of the administration units each comprising a said dose of iron-depleted lactoferrin. For a week-long treatment, one preferred product includes 7 of the administration units for daily administration over that period. For continued treatment of a chronic condition the number of administration units could exceed 100, for example to provide several months of supply.

67. The product of any one of paragraphs 1-66, comprising a said active pharmaceutical ingredient for treatment of bacterial vaginosis as a first active pharmaceutical ingredient for treatment of bacterial vaginosis and the product comprises at least one additional active pharmaceutical ingredient in a therapeutically effective dose for treatment of bacterial vaginosis, wherein the at least one additional pharmaceutical ingredient is not a said active pharmaceutical ingredient selected from the group consisting of essential metal, iron-depleted lactoferrin and lipocalin-2.

68. The product of paragraph 67, comprising a said additional active pharmaceutical ingredient is in an orally administrable form, optionally in a form selected from the group consisting of an oral capsule, oral pill or oral tablet.

69. The product of paragraph 68, wherein a said additional active pharmaceutical ingredient is in an intravaginally administrable form.

70. The product of paragraph 69, wherein the intravaginally administrable form comprising a said active pharmaceutical ingredient is selected from the group consisting of a vaginal suppository tablet, a vaginal capsule, a vaginal cream and a vaginal gel.

71. The product of either one of paragraph 69 or paragraph 70, wherein the intravaginally administrable form comprising a said additional active pharmaceutical ingredient is not a said administration unit comprising the first active pharmaceutical ingredient.

72. The product of either one of paragraph 69 or paragraph 70, wherein the intravaginal administrable form comprising a said additional active pharmaceutical ingredient is a said administration unit comprising the first active pharmaceutical ingredient.

73. The product of any one of paragraphs 67-72, wherein a said additional active pharmaceutical ingredient is an antibiotic.

74. The product of paragraph 73, wherein the antibiotic is selected from the group consisting of amoxicillin, ampicillin, ceftiofur, metronidazole, secnidazole, tinidazole, lincosamides, and clindamycin.

75. The product of any one of paragraphs 67-74, wherein the first active pharmaceutical ingredient comprises at least one member selected from the group consisting of the essential metal, preferably the manganese, and the iron-depleted lactoferrin, and preferably comprises the essential metal, more preferably the manganese.

76. The product of any one of paragraphs 1-75, wherein a said administration unit, preferably a vaginal suppository tablet, comprises a sugar or sugar alcohol, preferably a sugar for preferential metabolism relative to Lactobacillus iners of a Lactobacillus species other than Lactobacillus iners, and optionally selected from the group consisting of Lactobacillus crispatus, Lactobacillus jensenii, Lactobacillus gasseri, Lactobacillus helveticus, Lactobacillus rhamnosus, Lactobacillus reuteri and Lactobacillus casei and combinations thereof. Preferably, the sugar is at most poorly metabolized by Lactobacillus iners.

77. The product of paragraph 76, wherein the sugar or sugar alcohol is selected from the group consisting of lactose, sucrose, mannitol, maltitol, sorbitol, xylitol and combinations thereof.

78. The product of either one of paragraph 76 or paragraph 77, wherein the sugar or sugar alcohol is lactose.

79. The product of any one of paragraphs 76-783, wherein the administration unit comprising the sugar or sugar alcohol is a said administration unit comprising a said ingredient.

80. The product of any one of paragraphs 76-79, wherein the administration unit comprising the sugar or sugar alcohol is an administration unit comprising a said ingredient selected from the group consisting of the essential metal, preferably comprising the manganese, and the iron-depleted lactoferrin, and preferably the administration unit comprises the essential metal, and more preferably comprises the manganese.

81. The product of any one of paragraphs 1-80, comprising a probiotic of a Lactobacillus species other than Lactobacillus iners, and optionally selected from the group consisting of Lactobacillus crispatus, Lactobacillus Jensenii, Lactobacillus gasseri, Lactobacillus helveticus, Lactobacillus rhamnosus, Lactobacillus reuteri, Lactobacillus casei and combinations thereof.

82. The product of paragraph 81, wherein the probiotic is in an intravaginally administrable form, preferably a vaginal suppository tablet, comprising the probiotic.

83. The product of paragraph 82, wherein the intravaginally administrable form comprising the probiotic is not a said administration unit comprising a said ingredient.

84. The product of paragraph 82, wherein the intravaginally administrable form comprising the probiotic is a said administration unit comprising a said ingredient.

85. The product of any one of paragraphs 1-84, comprising a said administration unit comprising the lipocalin-2, and preferably bovine lipocalin-2.

86. The product of paragraph 85, wherein the administration unit comprising the lipocalin-2 is selected from the group consisting of a vaginal capsule, a vaginal suppository tablet, a vaginal cream and a vaginal gel, and preferably a vaginal suppository tablet.

87. The product of either one of paragraph 85 or paragraph 86, wherein the lipocalin-2 is in an amount in a range of from 0.3 to 30 milligrams in the administration unit comprising the lipocalin-2.

88. The product of any one of paragraph 85-87, wherein the administration unit lipocalin-2 also comprises lactoferrin, and preferably the lactoferrin is the iron-depleted lactoferrin.

89. The product of any one of paragraphs 85-88, comprising a plurality of the administration units, preferably vaginal suppository tablets, each comprising a said dose of lipocalin-2.

90. The product of paragraph 89, comprising at least 3 and preferably at least 5 of the administration units each comprising a said dose of lipocalin-2, preferably from 3 to 100 of the administration units each comprising a said dose of lipocalin-2, and sometimes from 7 to 10 of the administration units each comprising a said dose of lipocalin-2. For a week-long treatment, one preferred product includes 7 of the administration units for daily administration over that period. For continued treatment of a chronic condition the number of administration units could exceed 100, for example to provide several months of supply.

91. The product of any one of paragraphs 1-90, comprising a plurality of the administration units, and preferably wherein the plurality of the administration units comprises a plurality of vaginal suppository tablets.

92. The product of paragraph 91, comprising at least 3 and preferably at least 5 of the vaginal suppository tablets, preferably from 3 to 100 of the vaginal suppository tablets, and sometimes from 7 to 10 of the vaginal suppository tablets. For a week-long treatment, one preferred product includes 7 of the vaginal suppository tablets. For continued treatment of a chronic condition the number of the vaginal suppository tablets could exceed 100, for example to provide several months of supply.

93. The product of either one of paragraph 91 or paragraph 92, wherein the product is in the form of a kit comprising the plurality of the administration units contained in a common packaging enclosure.

94. The product of paragraph 93, wherein each said administration unit of the plurality of administration units is in a separate enclosure, preferably in a separate hermetically sealed enclosure, within the common packaging enclosure, for example the separate enclosures with administration units may be separate sealed cavities of a tray or blister pack comprising the plurality of the administration units.

95. The product of either one of paragraph 93 or 94, wherein the kit comprises a said vaginal applicator according to any one of paragraphs 21-24.

96. The product of any one of paragraphs 1-95, wherein a said administration unit is a vaginal suppository tablet comprising a gelling agent; and
wherein the vaginal suppository tablet is formulated to dissolve all of the said target component from the tablet over a time period of from 2 hours to 10 hours, and preferably from 3 to 10 hours, under the following conditions:
   a 2 milliliter sample of normal saline as a dissolution solution at 37° C. is placed into a 5 milliliter tube;
a tablet is placed in the tube containing the dissolution solution and the tube is sealed and secured to a rotary shaker;
the rotary shaker with the secured tubes is operated at a 30 degree tilt, speed of turns per minute and temperature setting of 37° C.;
the shaker is operated for 10 hours, pausing briefly for intermediate visual observation of tube contents at 0.5, 1, 2, 4 and 8 hours;
dissolution status of the tablet is record for each time point of 0.5, 1, 2, 4 and hours and the time point is recorded at which total tablet dissolution occurs or that total tablet dissolution did not occur during the 10 hour test period as determined by visual observation.

97. A method to treat a human for bacterial vaginosis, the method comprising:
intravaginal administration in water-soluble form for treatment of bacterial vaginosis of at least one active pharmaceutical ingredient selected from the group consisting of:
   essential metal, in a therapeutically effective dose of essential metal for treatment of bacterial vaginosis, the essential metal being selected from the group consisting of manganese at a therapeutically effective dose of manganese (preferably in a range of from 10 to 1000 micrograms of manganese), zinc at a therapeutically effective dose of zinc (preferably in a range of from 25 micrograms to 5000 micrograms of zinc) and combinations thereof;
   iron-depleted lactoferrin, in a therapeutically effective dose of iron-depleted lactoferrin for treatment of bacterial vaginosis and preferably in a range of from 50 to 500 milligrams of iron-depleted lactoferrin;
   lipocalin-2, in a therapeutically effective dose of lipocalin-2 for treatment of bacterial vaginosis and preferably in a range of from 0.3 to 30 milligrams of lipocalin-2; and
   combinations thereof. In some preferred implementations, the essential metal comprises manganese, alone or in combination with zinc.

98. A method to supplement vaginal bacterial health of a human, optionally to supplement nutritional immunity to bacterial infection associated with bacterial vaginosis, the method comprising:
intravaginal administration in a water-soluble form at least one vaginal health supplement ingredient selected from the group consisting of:
   essential metal in a health supplement dose of essential metal, the essential metal being selected from the group consisting of manganese in a health supplement dose of manganese (preferably in a range of from 10 to 1000 micrograms of manganese) and zinc at a health supplement dose of zinc (preferably in a range of from 25 to 5000 micrograms of zinc) and combinations thereof
   iron-depleted lactoferrin, in a health supplement dose of iron-depleted lactoferrin and preferably at a said dose of iron-depleted lactoferrin in a range of from 50 to 500 milligrams of iron-depleted lactoferrin;
   lipocalin-2, in a health supplement dose of lipocalin-2 and preferably in a range of from 0.3 to 30 milligrams of lipocalin-2; and
   combinations thereof. In some preferred implementations, the essential metal comprises manganese, alone or in combination with zinc.

99. The method of either one of paragraph 97 or paragraph 98, comprising the intravaginal administration of the essential metal, preferably comprising the manganese.

100. The method of paragraph 99, wherein the essential metal comprises the manganese and the dose of manganese is in a range having a lower limit selected from the group consisting of 10 micrograms, 20 micrograms, 30 micrograms or 50 micrograms and an upper limit selected from the group consisting of 1000 micrograms, 900 micrograms, 800 micrograms or 600 micrograms. One preferred range for the dose of manganese is from 20 micrograms to 900 micrograms, and one more preferred rage for the dose of manganese is from 30 micrograms to 800 micrograms.

101. The method of either one of paragraph 99 or paragraph 100, wherein the water-soluble form of the essential metal comprises a salt selected from the group consisting of manganese salt, zinc salt and combinations thereof.

102. The method of paragraph 101, wherein;
when the salt is manganese salt, the manganese salt is selected from the group consisting of manganese gluconate, manganese sulfate, manganese chloride, manganese citrate, manganese picolinate and combinations thereof, and with one particularly preferred manganese salt being zinc sulfate;
when the salt is zinc salt, the zinc salt is preferably zinc sulfate.

103. The method of any one of paragraphs 99-102, wherein the water-soluble form of the manganese comprises a chelate selected from the group consisting of a manganese chelate and a zinc chelate.

104. The method of paragraph 103, wherein the chelate is a manganese chelate and the manganese chelate is an amino acid chelate, preferably selected from the group consisting of manganese bisglycinate chelate, manganese glycinate chelate, manganese aspartate and combinations thereof.

105. The method of any one of paragraphs 99-104, wherein the essential metal comprises the manganese and the manganese in water-soluble form is not bound to the iron-depleted lactoferrin.

106. The method of any one of paragraphs 99-105, wherein the manganese is in the form of Mn(II).

107. The method of any one of paragraphs 99-106, wherein the intravaginal administration of the essential metal, preferably comprising the manganese, comprises intravaginal placement of an administration unit comprising the essential metal, and preferably comprising the manganese.

108. The method of paragraph 107, wherein the administration unit comprising the essential metal, preferably comprising the manganese, is a vaginal suppository tablet comprising the manganese.

109. The method of either one of paragraph 107 or paragraph 108, wherein the administration unit is a said administration unit comprising the essential metal, preferably the manganese, according to any one of paragraphs 1-117.

110. The method of any one of paragraphs 99-109, comprising a plurality of said intravaginal administrations of the essential metal, preferably comprising the manganese, with each said intravaginal administration comprising a said dose of essential metal, and preferably each said intravaginal administration comprises a said dose of manganese.

111. The method of paragraph 110, comprising a plurality of said intravaginal administrations of the manganese, and wherein the plurality of said intravaginal administrations of the manganese comprise a number of separate intravaginal administrations of a said dose of manganese in a range of from 10 to 1000 micrograms, preferably from 20 to 900 micrograms and more preferably from 30 to 800 micrograms.

112. The method of either one of paragraph 110 or paragraph 111, wherein the intravaginal administrations of all of the doses of essential metal, preferably comprising the manganese, occurs during a period of time for at least 3 days, preferably at least 5 days, more preferably at least 7 days, and optionally in a range of from 3 days to 100 days and with one preferred range being 7 days to 10 days. For continued treatment of a chronic condition the period of time may exceed 100 days.

113. The method of any one of paragraphs 110-112, wherein the intravaginal administrations of the plurality of said doses of the essential metal, and preferably comprising the manganese is at an administration frequency in a range of from 1 times per day to once every 3 days, with a preferred administration frequency of once per day.

114. The method of any one of paragraphs 97-113, comprising the intravaginal administration of the iron-depleted lactoferrin.

115. The method of paragraph 114, wherein the dose of iron-depleted lactoferrin is in a range of from 50 milligrams to 500 milligrams, and preferably in a range of from 200 milligrams to 400 milligrams.

116. The method of either one of paragraph 114 or paragraph 115, wherein the iron-depleted lactoferrin comprises a level of saturation with bound iron, in increasing order of preference, of not larger than 15 percent, not larger than 10 percent, not larger than 5 percent, not larger than 4 percent, not larger than 3 percent, not larger than 2 percent and most preferably not larger than 1 percent.

117. The method of paragraph 116, wherein the iron-depleted lactoferrin comprises a level of saturation with bound iron of at least 0.2 percent, and often at least 0.5 percent.

118. The method of any one of paragraphs 114-117, wherein the iron-depleted lactoferrin comprises a level of saturation with bound manganese of not more than 1 percent, preferably not larger than 0.5 percent and more preferably the iron-depleted lactoferrin is essentially in the absence of manganese bound to the iron-depleted lactoferrin.

119. The method of any one of paragraphs 114-118, wherein the iron-depleted lactoferrin is selected from the group consisting of human lactoferrin, bovine lactoferrin, recombinant lactoferrin and combinations thereof.

120. The method of any one of paragraphs 114-119, wherein the iron-depleted lactoferrin is bovine lactoferrin.

121. The method of any one of paragraphs 114-120, wherein the intravaginal administration of the iron-depleted lactoferrin comprises intravaginal placement of an administration unit comprising the iron-depleted lactoferrin.

122. The method of paragraph 121, wherein the administration unit comprising the iron-depleted lactoferrin is a vaginal suppository tablet comprising the iron-depleted lactoferrin.

123. The method of either one of paragraph 121 or paragraph 122, wherein the administration unit is a said administration unit comprising iron-depleted lactoferrin according to any one of paragraphs 1-117.

124. The method of any one of paragraphs 114-123, comprising a plurality of said intravaginal administrations of the iron-depleted lactoferrin, with each said intravaginal administration comprising a said dose of iron-depleted lactoferrin.

125. The method of paragraph 124-, wherein the intravaginal administrations of all of the doses of iron-depleted lactoferrin occurs during a period of time of at least 3 days, preferably at least 5 days, more preferably at least 7 days, and optionally in a range of form 3 days to app days. For continued treatment of a chronic condition the period of time may exceed 100 days.

126. The method of either on of paragraph 124 or paragraph 125, wherein the intravaginal administrations of the plurality of said doses of the magnesium is at an administration frequency in a range of from 1 times per day to once every 3 days, with a preferred administration frequency of once per day.

127. The method of any one of paragraphs 97-126, comprising the intravaginal administration of the lipocalin-2, and preferably bovine lipocalin-2.

128. The method of paragraph 127, wherein the dose of lipocalin-2 is in a range of from 0.3 to 30 milligrams of lipocalin-2.

129. The method of either one of paragraph 127 or paragraph 128, wherein the intravaginal administration of the lipocalin-2 comprises intravaginal placement of an administration unit comprising the lipocalin-2.

130. The method of any one of paragraphs 127-129, wherein the administration unit comprising the lipocalin-2 is selected from the group consisting of a vaginal capsule, a vaginal suppository tablet, a vaginal cream and a vaginal gel, and preferably a vaginal suppository tablet.

131. The method of any one of paragraphs 127-130, wherein the administration unit lipocalin-2 also comprises lactoferrin, and preferably the lactoferrin is the iron-depleted lactoferrin.

132. The method of any one of paragraphs 127-131, wherein the administration unit comprising the lipocalin-2 is a said administration unit comprising lipocalin-2 according to any one of paragraphs 1-117.

133. The method of any one of paragraphs 97-132, comprising the intravaginal administration for contemporaneous effect at least two said ingredients selected from the group of the manganese, the iron-depleted lactoferrin and the lipocalin-2.

134. The method of paragraph 133, wherein the at least two said ingredients include the manganese and the iron-depleted lactoferrin.

135. The method of either one of paragraph 133 or paragraph 134, wherein the at least two said ingredients include the manganese and the lipocalin-2.

136. The method of any one of paragraphs 133-135, wherein the at least two said ingredients include the iron-depleted lactoferrin and the lipocalin-2.

137. The method of any one of paragraphs 97-136, comprising performing a said intravaginal administration during or within 30 days after an administration of an active pharmaceutical agent for treatment of bacterial vaginosis in a therapeutically effective dose for treatment of bacterial vaginosis, and wherein the active pharmaceutical agent is not a said ingredient selected from the group consisting of manganese, iron-depleted lactoferrin and lipocalin-2.

138. The method of paragraph 137, comprising performing the administration of the active pharmaceutical agent.

139. The method of either one of paragraph 137 or paragraph 138, wherein the active pharmaceutical agent is administered orally in an orally administrable form, and optionally the orally administrable form is selected from the group consisting of an oral capsule, oral pill or oral tablet.

140. The method of either one of paragraph 137 or paragraph 138, wherein the active pharmaceutical agent is administered intravaginally in an intravaginally administrable form.

141. The method of paragraph 140, wherein the intravaginally administrable form comprising the active pharmaceutical agent is selected from the group consisting of a vaginal suppository tablet, a vaginal capsule, a vaginal cream and a vaginal gel.

142. The method of any one of paragraphs 137-141, wherein a said active pharmaceutical agent is an antibiotic.

143. The method of paragraph 142, wherein the antibiotic is selected from the group consisting of amoxicillin, ampicillin, ceftiofur, metronidazole, secnidazole, tinidazole, lincosamides, and clindamycin.

144. The method of any one of paragraphs 137-143, wherein the human receives a plurality of administrations of the active pharmaceutical agent, and the performing a said intravaginal administration occurs before a first one in time of the plurality of administrations of the active pharmaceutical agent.

145. The method of any one of paragraphs 137-143, wherein the human receives a plurality of administrations of the active pharmaceutical agent, and the performing a said intravaginal administration occurs after a last one in time of the plurality of administrations of the active pharmaceutical agent.

146. The method of paragraph 145, wherein a last one in time of the plurality of said intravaginal administrations occurs after the last one in time of the plurality of said administrations of the active pharmaceutical agent.

147. The method of any one of paragraphs 97-146, comprising an additional essential metal for growth of at least one Lactobacillus species, optionally selected from the group consisting of Lactobacillus crispatus, Lactobacillus jensenii, Lactobacillus gasseri, Lactobacillus helveticus, Lactobacillus rhamnosus, Lactobacillus reuteri and Lactobacillus casei, wherein the additional essential metal is copper, and wherein the administration of the additional essential metal occurs within a time period range to provide contemporaneous activity with a said ingredient administered by the intravaginal administration.

148. The method of paragraph 147, wherein the administration of the essential metal comprises administering a dose of copper.

149. The method of paragraphs 148, wherein the additional essential metal is intravaginally administered during the administration of the additional essential metal.

150. The method of paragraph 149, wherein the additional essential metal is intravaginally administered during the intravaginal administration of a said ingredient.

151. The method of paragraph 149, wherein the additional essential metal is intravaginally administered during the intravaginal administration of the said ingredient in an administration unit, preferably a vaginal suppository tablet, comprising both the additional essential metal and a said ingredient.

152. The method of paragraph 151, wherein the administration unit comprising both the additional essential metal and a said ingredient is an administration unit according to any one of paragraphs 1-96.

153. The method of any one of paragraphs 97-152, wherein the intravaginal administration comprises intravaginal placement of a vaginal suppository tablet comprising the manganese and the vaginal suppository tablet completely dissolves in the vaginal environment over a time period of not less than 12 hours, and preferably within 12 to 36 hours, following the intravaginal placement.

154. The product of any one of paragraphs 1-96, wherein a said administration unit, and preferably a vaginal suppository tablet, comprises an additional essential metal for growth of at least one Lactobacillus species, and optionally selected from the group consisting of Lactobacillus crispatus, Lactobacillus jensenii, Lactobacillus gasseri, Lactobacillus helveticus, Lactobacillus rhamnosus, Lactobacillus reuteri and Lactobacillus casei, wherein the additional essential metal is copper.

155. The product of paragraph 154, wherein the administration unit comprises a dose of copper at least as large as 10 micrograms of copper.

156. The product of paragraph 155, wherein the dose of copper is up to 1000 micrograms of copper.

157. A product of any one of paragraphs 154-156, comprising a plurality of the administration units comprising the additional essential metal.

158. A product according to any one of paragraphs 154-157, wherein each said administration unit comprising the additional essential metal is in a vaginal suppository tablet according to paragraph 96.

159. A slow release intravaginal suppository tablet for intravaginal delivery of an active pharmaceutical ingredient for treatment of a vaginal condition in a human, the intravaginal suppository tablet comprising:
target material for intravaginal delivery, wherein the target material comprises an active pharmaceutical ingredient for treatment of a vaginal condition; and
a gelling agent; and
   formulated to dissolve all of the said ingredient from the tablet, and preferably to dissolve the tablet, over a time period of from 20 minutes to 90 minutes, and preferably from 30 minutes to 90 minutes, under the conditions of Test Protocol A and/or over a time period of 2 hours to 10 hours, and preferably from 3 to 10 hours, under the conditions of Test Protocol B.

160. A slow release intravaginal suppository tablet for intravaginal delivery of a vaginal supplement for vaginal bacterial health in a human, optionally to supplement nutritional immunity to bacterial infection associated with bacterial vaginosis, the intravaginal suppository tablet comprising:
target material for intravaginal delivery, wherein the target material comprises a vaginal supplement ingredient for vaginal bacterial health; and
a gelling agent; and
formulated to dissolve all of the said ingredient from the tablet, and preferably to dissolve the tablet, over a time period of from 20 minutes to 90 minutes, and preferably from 30 minutes to 90 minutes, under the conditions of Test Protocol A and/or over a time period of 2 hours to 10 hours under the conditions of Test Protocol B

161. The product or intravaginal suppository tablet of any one of paragraphs 96 and 158-160, wherein the gelling agent is a bioadhesive gelling agent.

162. The product or intravaginal suppository tablet of paragraph 161, wherein the gelling agent is selected from the group consisting of acacia, alginic acid, bentonite, carbomers, carboxymethyl cellulose, ethylcellulose, gelatin, hydroxyethyl cellulose, hydroxypropylcellulose, magnesium aluminum silicate, methylcellulose, poloxamers, polyvinyl alcohol, sodium alginate, tragacanth, xanthan gum, poloxamer 407, poloxamer 188, polyvinylpyrrolidone, copovidone (vinylpyrrolidone-vinyl acetate copolymer) (e.g., Kollidon® VA 64, BASF), celluloses, chitosans, mucin glycoproteins, trefoil peptides, polycarbophils and combinations thereof, and preferably with the gelling agent selected from the group consisting of poloxamer 407, poloxamer 188, polyvinylpyrrolidone, copovidone (e.g., Kollidon® VA 64, BASF), celluloses, chitosans, mucin glycoproteins, trefoil peptides, polycarbophils and combinations thereof, and more preferably with the gelling agent selected from the group consisting of celluloses, polyvinylpyrrolidone copovidone (e.g., Kollidon® VA 64, BASF), and poloxamer 407.

163. The product or intravaginal suppository tablet of any one of paragraphs 96 and 158-162, comprising the gelling agent at a concentration in a range of from 0.5 to 10 weight percent, and with one more preferred range for the concentration of the gelling agent being from 1 to 5 weight percent and an even more preferred range being from 2 to 5 weight percent.

164. The product or intravaginal suppository tablet of any one of paragraphs 96 and 158-163, wherein the vaginal suppository tablet is either in the in the absence of a disintegrant or contains a disintegrant only on one side of a bilayer tablet.

165. The product or intravaginal suppository tablet of any one of paragraphs 96 and 158-164, wherein the vaginal suppository tablet is essentially in the absence of each of the following: crospovidone (cross-linked polyvinylpyrrolidone).

166. The product or intravaginal suppository tablet of paragraph 165, wherein the vaginal suppository tablet comprises from 0.01 to 20 weight percent of a binder dispersed in the vaginal suppository tablet, and with one more preferred range for the concentration of the binder being from 0.05 to 10 weight percent.

167. The product or intravaginal suppository tablet of paragraph 166, wherein the binder is selected from the group consisting of lactose, sucrose, mannitol, microcrystalline cellulose, maltitol, sorbitol, xylitol, water-soluble hydroxyalkyl celluloses, polyvinylpyrrolidone, povidone, xanthan gum, celluloses (hydroxypropylmethylcellulose, hydroxypropyl cellulose) gelatin, starch, polyethylene glycol and combinations thereof, and preferably with the binder selected from the group consisting of lactose, sucrose, mannitol, microcrystalline cellulose, maltitol, sorbitol, xylitol and combinations thereof, and with one particularly preferred binder being mannitol.

168. The product or intravaginal suppository tablet of any one of paragraphs 96 and 158-167, wherein the vaginal suppository tablet comprises up to 80 weight percent filler dispersed in the vaginal suppository tablet, and with one more preferred range for the concentration of the filler being up to 40 weight percent.

169. The product or intravaginal suppository tablet of paragraph 168, wherein the filler comprises filler material selected from the group consisting of lactose, sucrose, mannitol, magnesium stearate, glucose, plant cellulose, calcium carbonate and combinations thereof, and preferably with the filler material being mannitol, which is not a good bacterial foof source.

170. The product or intravaginal suppository tablet of any one of paragraphs 96 and 158-169, wherein the vaginal suppository tablet comprises from 0 to 50 weight percent sugar or sugar alcohol dispersed in the vaginal suppository tablet, and with one more preferred range for the concentration of the sugar or sugar alcohol being from 20 to 40 weight percent.

171. The product or intravaginal suppository tablet of paragraph 170, wherein the sugar or sugar alcohol is selected from the group consisting of lactose, sucrose, mannitol, maltitol, sorbitol, xylitol and combinations thereof, and preferably with the filler material selected from the group consisting of lactose, sucrose, mannitol and combinations thereof, and with one particularly preferred filler material being lactose.

172. The product or intravaginal suppository tablet of any one of paragraphs 96 and 158-171, wherein the vaginal suppository tablet comprises from 0.25 to 5 weight percent lubricant dispersed in the vaginal suppository tablet, and with one more preferred range for the concentration of the lubricant being from 0.5 to 3 weight percent.

173. The product or intravaginal suppository tablet of paragraph 172, wherein the lubricant is selected from the group consisting of metallic salts of fatty acids (Magnesium stearate, calcium stearate, and zinc Stearate), fatty acids (e.g. stearic acid) fatty acid esters (e.g. glyceride esters (glyceryl monostearate, glyceryl tribehenate, and glyceryl dibehenate) and sugar esters (e.g., sorbitan monostearate and sucrose monopalmitate) and combinations thereof, and preferably with the lubricant selected from the group consisting of fatty acid esters and combinations thereof and combinations thereof, and with one particularly preferred lubricant being glyceryl dibehenate.

174. The product or intravaginal suppository tablet of any one of paragraphs 96 and 158-174, wherein the vaginal suppository tablet comprises from 0 to 5 weight percent glidant dispersed in the vaginal suppository tablet, and with one more preferred range for the concentration of the glidant being from 0.25 to 2 weight percent.

175. The product or intravaginal suppository tablet of paragraph 174, wherein the glidant is selected from the group consisting of talc, colloidal silicon dioxide, magnesium stearate, silica and combinations thereof, and with one particularly preferred glidant being silicon dioxide, and preferably a colloidal silicon dioxide, such as for example fumed silica.

176. A product comprising manganese for use in the treatment of bacterial vaginosis, and optionally the product is according to any one of paragraphs 1-96.

177. A product comprising manganese for use in the supplementation of vaginal bacterial health, and optionally the product is according to any one of paragraphs 1-96.

178. A product comprising iron-depleted lactoferrin for use in the treatment of bacterial vaginosis, and optionally the product is according to any one of paragraphs 1-96.

179. A product comprising iron-depleted lactoferrin for use in the supplementation of vaginal bacterial health, and optionally the product is according to any one of paragraphs 1-96.

180. A product comprising lipocalin-2 for use in the treatment of bacterial vaginosis, and optionally the product is according to any one of paragraphs 1-96.

181. A product comprising lipocalin-2 for use in the supplementation of vaginal bacterial health, and optionally the product is according to any one of paragraphs 1-96.

182. A product comprising zinc for use in the treatment of bacterial vaginosis, and optionally the product is according to any one of paragraphs 1-96.

183. A product comprising zinc for use in the supplementation of vaginal bacterial health, and optionally the product is according any one of paragraphs 1-96.

184. A product comprising manganese and iron-depleted lactoferrin for use in the treatment of bacterial vaginosis, and optionally the product is according to any one of paragraphs 1-96.

185. A product comprising manganese and iron-depleted lactoferrin for use in the supplementation of vaginal bacterial health, and optionally the product is according to any one of paragraphs 1-96.

186. A product comprising any two or all three active ingredient selected from the group consisting of manganese, iron-depleted lactoferrin and lipocalin-2 for use in the treatment of bacterial vaginosis, and optionally the product is according to any one of paragraphs 1-96.

187. The product of any one of paragraphs 176-186, comprising a sugar for preferential metabolism relative to Lactobacillus iners of at least one Lactobacillus species other than Lactobacillus iners bacterial member selected from the group consisting of Lactobacillus crispatus, Lactobacillus jensenii, Lactobacillus gasseri, Lactobacillus helveticus, Lactobacillus rhamnosus, Lactobacillus reuteri and Lactobacillus casei and combinations thereof, and preferably the sugar is at most poorly metabolized by Lactobacillus iners, and more preferably the sugar is lactose.

188. The product of any one of paragraphs 176-187, comprising iron-depleted bovine lactoferrin.

189. The product of any one of paragraphs 176-188, comprising an antibiotic for treatment of bacterial vaginosis.

190. The product of any one of paragraphs 176-188, comprising probiotic of a Lactobacillus other than lactobacillus iners.

191. The product of any one of paragraphs 176-190, in the form of a vaginal suppository tablet.

192. The product of any one of paragraphs 176-191, in the form of a plurality of vaginal suppository tablets for periodic administration as part of a treatment regimen, each said vaginal suppository tablet comprising one or more of the ingredients for treatment of bacterial vaginosis and/or for supplementation of vaginal bacterial health.

A1. A slow release therapeutic composition for vaginal drug delivery, the composition comprising:
at least one active pharmaceutical ingredient;
a binder;
a filler; and
a gelling agent.

A2. The composition of paragraph A1, wherein the active pharmaceutical ingredient is selected from the group consisting of proteins, peptides, minerals, trace elements, antibiotics, antifungals, anti-inflammatories, cytokines, hormones, anesthetics, analgesics and fragments thereof.

A3. The composition of paragraph A1, wherein the active pharmaceutical ingredient is selected from the group consisting of lactoferrin, bovine lactoferrin, apolactoferrin, bovine apolactoferrin, manganese, lipocalin-2, and combinations thereof for the treatment of bacterial vaginosis.

A4. The composition of any one of paragraphs paragraph A1-A3, further comprising excipients selected from the group consisting of a glidant and a lubricant.

A5. The composition of any one of paragraphs A1-A3, wherein the composition is in a tablet or a suppository form; administered intravaginally and dissolving slowly following contact with vaginal fluid.

A6. The composition of any one of paragraphs A1 and A3-A6, wherein the active pharmaceutical ingredient is bovine lactoferrin.

A7. The composition of any one of paragraphs A1-A6, wherein the composition is administered before, during or after administration of an antibiotic and the combination of the composition and the antibiotic provide a synergistic therapy.

A8. The composition of paragraph A7, wherein the antibiotic is selected from the group consisting of clindamycin, amoxicillin, ampicillin, ceftiofur, metronidazole, tinidazole, secnidazole or combinations thereof.

A9. The composition of any one of paragraphs A3-A8-, wherein the active pharmaceutical ingredient is bovine lactoferrin and the composition further comprises manganese as a second active pharmaceutical ingredient.

A10. The composition of any one of paragraphs A3-A5 and A7-A8, A3, wherein the pharmaceutical ingredient is manganese.

A11. The composition of any one of paragraphs A3-A5 and A7-A8, wherein the pharmaceutical ingredient is lipocalin-2.

A12. The composition of any one of paragraphs A3-A8 and A10, wherein the composition further comprises lipocalin-2 as a second active pharmaceutical ingredient.

A13. The composition of any one of paragraphs A3-A5 and A7-A8, wherein the pharmaceutical ingredient is apo lactoferrin.

A14. The composition of any one of paragraphs A3-A8 and A10, wherein the composition further comprises apolactoferrin as a second active pharmaceutical ingredient.

A15. The composition of paragraph A11, wherein the composition further comprises apolactoferrin as a second active pharmaceutical ingredient.

A16. A method for delivering a slow release therapeutic composition to the vagina, the method comprising administering a composition comprising:
at least one active pharmaceutical ingredient;
a binder;
a filler; and
a gelling agent.

A17. The method of paragraph A16, wherein the active pharmaceutical ingredient is selected from the group consisting of lactoferrin, bovine lactoferrin, apolactoferrin, bovine apolactoferrin, manganese, lipocalin-2, an antibiotic and combinations thereof for treating bacterial vaginosis.

A18. The method of paragraph A17, wherein the active pharmaceutical ingredient is bovine lactoferrin and the composition further comprising excipients selected from the group consisting of a glidant and a lubricant.

A19. The method of any one of paragraphs A16-A18, wherein the administering of the composition is before, during or after administration of an antibiotic.

A20. A therapeutic composition for vaginal drug delivery, the composition comprising: at least one active pharmaceutical ingredient selected from the group consisting of lactoferrin, bovine lactoferrin, manganese, apolactoferrin, lipocalin-2 and combinations thereof;
a binder; and
a filler for treatment of bacterial vaginosis.

A21. The therapeutic composition of paragraph A20, wherein the composition is administered before, during or after administration of an antibiotic.

A22. A method for delivering a therapeutic composition to the vagina for treatment of bacterial vaginosis, the method comprising administering a composition comprising:
at least one active pharmaceutical ingredient selected from the group consisting of manganese, lactoferrin, bovine lactoferrin, apolactoferrin and lipocalin-2 and combinations thereof;
a binder;
a filler and
delivering the therapeutic composition to the vaginal mucosa.

A23. The method of paragraph A22, wherein the composition is administered before, during or after administration of an antibiotic.

A24. A kit comprising at least one therapeutic composition and optionally instructions that said composition is to be applied to vaginal mucosa daily for treatment of bacterial vaginosis, wherein the composition comprises:
at least one active pharmaceutical ingredient;
a binder;
a filler and
a gelling agent.

A25. The kit of paragraph A24, wherein the active pharmaceutical ingredient is selected from the group consisting of lactoferrin, bovine lactoferrin, apolactoferrin, bovine apo-lactoferrin, manganese, lipocalin-2, and combinations thereof.

A26. The kit of either one of paragraph A24 or paragraph A25, further comprising at least one antibiotic and optionally instructions that said antibiotic is to be administered before, during or after administration of the therapeutic composition for treatment of bacterial vaginosis.

A27. A method for preventing pre-term labor comprising administering a slow release therapeutic composition to the vaginal mucosa comprising:
at least one active pharmaceutical ingredient;
a binder;
a filler;
a gelling agent;
and wherein the active pharmaceutical ingredient is selected from the group consisting of lactoferrin, bovine lactoferrin, apolactoferrin, bovine apolactoferrin, manganese, lipocalin-2, an antibiotic and combinations thereof.

The terms “comprising”, “containing”, “including” and “having”, and grammatical variations of those terms, are intended to be inclusive and nonlimiting in that the use of such terms indicates the presence of some condition or feature, but not to the exclusion of the presence also of any other condition or feature. The use of the terms “comprising”, “containing”, “including” and “having”, and grammatical variations of those terms in referring to the presence of one or more components, subcomponents or materials, also include and is intended to disclose the more specific embodiments in which the term “comprising”, “containing”, “including” or “having” (or the variation of such term) as the case may be, is replaced by any of the narrower terms “consisting essentially of” or “consisting of” or “consisting of only” (or the appropriate grammatical variation of such narrower terms). For example, a statement that something “comprises” a stated element or elements is also intended to include and disclose the more specific narrower embodiments of the thing “consisting essentially of” the stated element or elements, and the thing “consisting of” the stated element or elements. Examples of various features have been provided for purposes of illustration, and the terms “example”, “for example” and the like indicate illustrative examples that are not limiting and are not to be construed or interpreted as limiting a feature or features to any particular example. The term “at least” followed by a number (e.g., “at least one”) means that number or more than that number. The term at “at least a portion” means all or a portion that is less than all. The term “at least a part” means all or a part that is less than all.

What is claimed is:

1. A product for use to supplement vaginal microflora bacterial health in a human, optionally for treatment of bacterial vaginosis, the product comprising:
at least one administration unit configured for intravaginal administration, wherein a said administration unit comprises manganese at a therapeutically effective dose of manganese for treatment of bacterial vaginosis.

2. The product of claim 1, wherein the dose of manganese is in a range of from to 1000 micrograms of manganese.

3. The product of claim 1, comprising a plurality of the administration units each comprising a said dose of manganese.

4. The product of claim 3, comprising at least 3 of the administration units comprising a said dose of manganese.

5. The product of claim 3, comprising from 3 to 10 of the administration units comprising a said dose of manganese.

6. The product of claim 1, wherein the manganese is not bound to lactoferrin.

7. The product of claim 1, comprising an iron scavenger at a dose of iron scavenger for vaginal microflora bacterial health supplement and/or in a therapeutically effective amount for treatment of bacterial vaginosis, wherein the iron scavenger is contained in a form for intravaginal administration.

8. The product of claim 7, wherein the iron scavenger comprises lactoferrin.

9. The product of claim 8, wherein the lactoferrin is iron-depleted lactoferrin, wherein the iron-depleted lactoferrin comprises a level of saturation with bound iron of not larger than 4 percent.

10. The product of claim 9, wherein the iron-depleted lactoferrin is bovine lactoferrin.

11. The product of claim 10, wherein the form for intravaginal administration is a vaginal suppository tablet.

12. The product of claim 11, wherein the administration unit comprising the dose of manganese is the vaginal suppository tablet comprising the iron-depleted bovine lactoferrin.

13. The product of claim 12, comprising a plurality of the administration units each comprising a said dose of iron-depleted lactoferrin and a said dose of manganese.

14. The product of claim 13, wherein:
the dose of manganese is in a range of from 10 to 1000 micrograms; and
the dose of the iron-depleted lactoferrin is in a range of from 50 to 500 milligrams.

15. The product of any one of claims 1-14, comprising zinc.

16. The product of claim 15, wherein the administration unit comprising the manganese comprises the zinc at a dose of zinc for vaginal bacterial health supplementation and/or in a therapeutically effective amount for treatment of bacterial vaginosis.

17. The product of claim 16, wherein the dose of zinc is in a range of form 25 micrograms to 5000 micrograms.

18. The product of any one of claims 1-14, wherein the product comprises lipocalin-2.

19. The product of claim 18, wherein the administration unit comprising the manganese comprises the lipocalin-2 at a dose of lipocalin-2 for vaginal bacterial health supplementation and/or in a therapeutically effective amount for treatment of bacterial vaginosis.

20. The product of claim 19, wherein the dose of lipocalin-2 is in a range of 0.3 to milligrams.

21. The product of any one of claims 1-14 comprising an antibiotic for treatment of bacterial vaginosis.

22. The product of any one of claims 1-14, comprising a sugar for metabolism of at least one Lactobacillus species other than Lactobacillus iners and which sugar is poorly metabolized by Lactobacillus iners relative to the at least one Lactobacillus species other than Lactobacillus iners.

23. The product of claim 22, wherein the sugar comprises lactose.

24. The product of claim 23, comprising a probiotic of at least one Lactobacillus species other than Lactobacillus iners.

25. The product of any one of claims 1-14, comprising a vaginal applicator configured to carry the administration unit during insertion into a vagina for intravaginal placement of the administration unit.

26. The product of claim 25, comprising a plurality of said administration units each received in a different one of a plurality of said vaginal applicators for intravaginal placement, and wherein:
each said vaginal applicator having a said administration unit received therein for intravaginal placement is individually enclosed in a separate enclosure; and
each said vaginal applicator and each said administration unit are contained in a common packaging enclosure.

27. The product of any one of claims 1-14, wherein:
the administration unit comprising the manganese is in a form of a vaginal suppository tablet;
the vaginal suppository tablet comprising the manganese is formulated to dissolve from the vaginal suppository tablet all of the manganese over a time period of from 20 to 90 minutes according to dissolution test procedure of U.S. Pharmacopeia <711> into 900 mL of dissolution medium of 0.2M acetate buffered solution at pH 4.5±0.05 and 37±0.5° C. in a basket dissolution apparatus with 1 liter vessel volume and mixing speed of 75 revolutions per minute, with 5 mL syringe samples of dissolution medium collected at intervals of 15, 30, 45, 60, 90 and 120 minutes and analyzed to determine extent of dissolution of target component; and
the vaginal suppository tablet comprising the manganese is formulated to dissolve from the vaginal suppository tablet all of each said ingredient included in the vaginal suppository tablet over a time period of from 2 to 10 hours under the following test conditions:
a 2 milliliter sample of normal saline as a dissolution solution at 37° C. is placed into a 5 milliliter tube;
a tablet is placed in the tube containing the dissolution solution and the tube is sealed and secured to a rotary shaker;
the rotary shaker with the secured tubes is operated at a 30 degree tilt, speed of turns per minute and temperature setting of 37° C.;
the shaker is operated for 10 hours, pausing briefly for intermediate visual observation of tube contents at 0.5, 1, 2, 4 and 8 hours;
dissolution status of the tablet is record for each time point of 0.5, 1, 2, 4 and hours and the time point is recorded at which total tablet dissolution occurs or that total tablet dissolution did not occur during the 10 hour test period as determined by visual observation.

28. A method to treat a human for bacterial vaginosis, the method comprising:
intravaginal administration in water-soluble form of manganese in a therapeutically effective dose for treatment of bacterial vaginosis.

29. The method of claim 28, wherein the intravaginal administration is a prophylactic administration to a said human not having bacterial vaginosis at the time of the intravaginal administration.

30. The method of claim 28, wherein the intravaginal administration is to a said human exhibiting one or more symptoms associated with bacterial vaginosis at the time of the intravaginal administration.

31. The method of claim 28, wherein the dose of manganese is in a range of from microgram to 1000 micrograms of manganese.

32. The method of claim 28, wherein the dose of manganese is in a range of from to 800 micrograms of manganese.

33. The method of any one of claims 28-32, comprising a plurality of said intravaginal administrations of the manganese with each said intravaginal administration comprising a said dose of the manganese.

34. The method of claim 33, wherein the plurality of said intravaginal administrations comprise a number of separate said intravaginal administrations of at least 3.

35. The method of claim 34, wherein the intravaginal administrations are at an administration frequency of no more frequent than once per day

36. The method of any one of claims 28-32, comprising intravaginal administration of zinc, in a therapeutically effective dose of zinc for treatment of bacterial vaginosis, for contemporaneous intravaginal effect with the manganese.

37. The method of claim 36, wherein the dose of zinc is in a range of from 25 to 5000 micrograms of zinc.

38. The method of any one of claims 28-32, comprising intravaginal administration of iron-depleted bovine lactoferrin, in a therapeutically effective dose of iron-depleted bovine lactoferrin for treatment of bacterial vaginosis, for contemporaneous intravaginal effect with the manganese of the dose of manganese.

39. The method of claim 38, wherein the dose of ir38-depleted bovine lactoferrin is in a range of from 50 to 500 milligrams of iron-depleted lactoferrin.

40. The method of claim 39, wherein the iron-depleted bovine lactoferrin comprises a level of saturation with bound iron of not larger than 4 percent.

41. The method of any one of claims 28-32, comprising intravaginal administration of lipocalin-2, in a therapeutically effective dose of lipocalin-2 for treatment of bacterial vaginosis, for contemporaneous intravaginal effect with the manganese of the dose of manganese.

42. The method of claim 41, wherein the dose of lipocalin-2 is in a range of from 0.3 to 30 milligrams of lipocalin-2.

43. The method of any one of claims 28-32, comprising performing a said intravaginal administration of manganese within a time period range of during to 30 days after administration of an antibiotic for treatment of bacterial vaginosis.

44. The method of claim 43, wherein the administration of the antibiotic is an oral administration of the antibiotic.

45. The method of claim 44, wherein the administration of the antibiotic is an intravaginal administration of the antibiotic.

46. The method of any one of claims 28-32, comprising intravaginal administration of a probiotic of a Lactobacillus species other than Lactobacillus iners.

47. The method of claim 46, wherein the probiotic is intravaginally administered in an administration unit also comprising a said dose of manganese.

48. The method of any one of claims 28-32, wherein the intravaginal administration comprises intravaginal placement of a vaginal suppository tablet comprising the manganese and the vaginal suppository tablet completely dissolves in the vaginal environment within 12 to 36 hours following the intravaginal placement.

49. A method to treat a human for bacterial vaginosis, the method comprising:
providing a product of any one of claims 1-14; and
intravaginal administration of a said administration unit of the product.
